(19) United States
(12) Patent Application Publication
(10) Pub. No.: US 2025/0191679 A1
(43) Pub. Date: Jun. 12, 2025

(54) POLYGENIC RISK SCORE FOR CORONARY HEART DISEASE, CONSTRUCTION METHOD THEREFOR, AND APPLICATION THEREOF IN COMBINATION WITH CLINICAL RISK ASSESSMENT

(71) Applicant: Fuwai Hospital, Chinese Academy of Medical Sciences and Peking Union Medical College, Beijing City (CN)

(72) Inventors: Dongfeng Gu, Beijing City (CN); Xiangfeng Lu, Beijing City (CN); Jianfeng Huang, Beijing City (CN); Jianxin Li, Beijing City (CN); Fangchao Liu, Beijing City (CN)

(21) Appl. No.: 18/564,480

(22) PCT Filed: May 26, 2022
(86) PCT No.: PCT/CN2022/095221

§ 371 Date: Jun. 17, 2024

(30) Foreign Application Priority Data
May 26, 2021 (CN) 202110579226.1
May 26, 2021 (CN) 202110579230.8

(51) Int. Cl. G16B 20/20; A61B 5/00; C12Q 1/6883; G16H 10/40; G16H 50/30

(52) U.S. Cl. CPC G16B 20/20; C12Q 1/6883; G16H 10/40; G16H 50/30; A61B 5/7275; C12Q 2600/118; C12Q 2600/156

(57) ABSTRACT

A polygenic risk score (PRS) for coronary artery disease, a construction method therefor, and an application thereof in combination with clinical risk assessment. The present invention first provides an application of a reagent for detecting individual information in preparation of a detection device for assessing the onset risk of the coronary artery disease, wherein the individual information comprises 311 CAD-related single nucleotide polymorphic sites, and the individual information preferably also comprises one or more of BP, BMI, DM, TC and Stroke-related single nucleotide polymorphic sites. The present invention further provides a method for constructing a comprehensive metaPRS for the coronary artery disease. In the present invention, the PRS and the conventional clinical risk factor score are further integrated, such that re-layering of the onset risk of the coronary artery disease can be realized. The present invention is of great significance to primary prevention of the coronary artery disease.

### TECHNICAL FIELD

The present invention relates to a polygenic risk score (PRS) for coronary artery disease and a method of establishing the same and the use thereof in combination with clinical risk evaluation, and specifically the polygenic risk score for coronary artery disease comprises a PRS for coronary artery disease and a comprehensive score metaPRS for a plurality of subphenotypes of coronary artery disease.

### BACKGROUND

The onset and development of cardiovascular disease (CVD) is influenced by a combination of genetic and environmental factors. Risk prediction and evaluation play a crucial role in the primary prevention of cardiovascular disease. Genetic factors, as stable and quantifiable lifelong markers, have long been expected to be used in risk evaluation of diseases to facilitate precise prevention of cardiovascular diseases. Over the past decade, genome-wide association studies have successfully identified hundreds of regions significantly associated with coronary artery disease and coronary artery disease-related phenotypes (blood lipid levels, blood pressure, type 2 diabetes, and BMI). Recently, a polygenic risk score (PRS) for coronary artery disease that integrates information from multiple genetic variations has been successfully developed and used to evaluate the clinical efficacy of coronary artery disease risk prediction (Eur Heart. J. 37, 561-567 (2016); Nat. Genet. 50, 1219-1224 (2018); J. Am. Coll. Cardiol. 72, 1883-1893 (2018); Eur Heart. J. 37, 3267-3278 (2016); Jama 323, 627-635(2020); Jama 323, 636-645, (2020); JAMA Cardiol . . . 3, 693-702 (2018); N. Engl. J Med 375, 2349-2358 (2016)). However, almost all of these genetic scores were established based on European populations, and the differences in the frequency of variant loci and in the pattern of linkage disequilibrium among different populations have led to the fact that the scores from European populations cannot be used in East Asian and Chinese populations. In addition, differences in lifestyle, other risk factors, and potential gene-environment interactions among different populations also contribute to this heterogeneity. Some studies have reported that the predictive effect of these genetic scores is significantly reduced in predictive efficacy in other ethnic groups.

In addition, significant differences in environmental risk factors (lifestyle, dietary nutrition, and behavioral factors) and gene-environment interactions among different populations may also contribute to differential risks of coronary artery disease and benefits of intervention. Integration of polygenic risk scores and traditional risk factor scores to achieve re-stratification of the risk of developing coronary artery disease is important for primary prevention of coronary artery disease.

### SUMMARY OF THE INVENTION

One object of the present invention is to provide coronary artery disease-related single nucleotide polymorphism loci and a system for evaluating the risk of developing the disease applicable to an East Asian population.

Another object of the present invention is to provide a method for establishing a polygenic risk score (evaluation system) for coronary artery disease.

Upon extensive studies as well as detection and analysis tests in practice, the inventors have identified a group of coronary artery disease risk-related genes associated with East Asian populations which include 311 CAD-related single nucleotide polymorphism loci, and the risk of developing coronary artery disease in East Asian populations can be well evaluated by detecting these CAD-related single nucleotide polymorphism loci. The present invention further identifies BP, BMI, DM, TC, and Stroke-associated single nucleotide polymorphism loci, and the risk of developing coronary artery disease in East Asian populations can be better evaluated by further detecting one or more of these associated single nucleotide polymorphism loci.

Specifically, in one aspect, the present invention provides the use of a reagent for obtaining an individual's information in the manufacture of a device for evaluating the risk of developing coronary artery disease, wherein the individual's information comprises the following single nucleotide polymorphism locus information:

CAD-associated single nucleotide polymorphism loci: rs10064156, rs10071096, rs10093110, rs10096633, rs10139550, rs10237377, rs10260816, rs10267593, rs1027087, rs10278336, rs10455782, rs10503675, rs10512861, rs10513801, rs10745332, rs10757274, rs10773003, rs10842992, rs10846744, rs10857147, rs10890238, rs10953541, rs10968576, rs11030104, rs11057830, rs11067762, rs11077501, rs11099493, rs11107829, rs11125936, rs11142387, rs1116357, rs11170820, rs11205760, rs11206510, rs11509880, rs11556924, rs11557092, rs115696548, rs11601507, rs11677932, rs1169288, rs1173766, rs11787792, rs11810571, rs11838267, rs11838776, rs11847697, rs11911017, rs12175867, rs12214416, rs12445022, rs12463617, rs1250229, rs12524865, rs12597579, rs12603327, rs12692735, rs12718465, rs12740374, rs12801636, rs12932445, rs12936587, rs12970066, rs130071, rs13078807, rs1317507, rs13209747, rs1321309, rs13306194, rs13359291, rs1344653, rs1351525, rs13723, rs1378942, rs1412444, rs1421085, rs148910227, rs1496653, rs151193009, rs1514175, rs1535500, rs1552224, rs1555543, rs1563788, rs1591805, rs16849225, rs16858082, rs16986953, rs16990971, rs16999793, rs17030613, rs17035646, rs17080102, rs17087335, rs17135399, rs17249754, rs173396, rs17358402, rs17381664, rs174547, rs17465637, rs17477177, rs17514846, rs17612742, rs17678683, rs17695224, rs1800588, rs181360, rs1861411, rs1868673, rs1870634, rs1887320, rs1892094, rs191835914, rs1976041, rs2000999, rs200990725, rs2021783, rs2057291, rs2066714, rs2068888, rs2075260, rs2075291, rs2107595, rs2128739, rs2144300, rs2145598, rs2156552, rs216172, rs2200733, rs2213732, rs2229383, rs2230808, rs2237896, rs2240736, rs2268617, rs2297991, rs2303790, rs2328223, rs2383208, rs2531995, rs2535633, rs2571445, rs2575876, rs261967, rs2782980, rs2815752, rs2819348, rs2820443, rs2925979, rs2954029, rs29941, rs3120140, rs3129853, rs3130501, rs326214, rs351855, rs35332062, rs35337492, rs35444, rs36096196, rs3775058, rs3785100, rs3809128, rs3827066, rs3846663, rs3887137, rs4129767, rs4148008, rs4266144, rs4302748, rs4377290, rs4409766, rs4410190, rs4420638, rs4468572, rs459193, rs4593108, rs4613862, rs46522, rs4713766, rs4719841, rs4731420, rs4735692, rs4752700, rs4766228, rs4776970, rs4788102, rs4812829, rs4821382, rs4836831, rs4845625, rs4883263, rs4911495, rs4917014, rs4918072, rs499974, rs515135, rs5215, rs556621, rs56062135, rs56289821, rs56336142, rs574367, rs582384, rs590121, rs6038557, rs6065311, rs633185, rs635634, rs6494488, rs651821, rs663129, rs667920, rs6700559, rs671, rs6725887, rs6795735, rs6804922, rs6807945, rs6808574, rs6813195, rs6818397, rs6829822, rs6882076, rs6905288, rs6909752, rs6960043, rs699, rs6997340, rs702485, rs7087591, rs7120712, rs7178572, rs7185272, rs7199941, rs7202877, rs7206541, rs7208487, rs7225581, rs7258445, rs72654473, rs72689147, rs73015714, rs7304841, rs7306523, rs73069940, rs738409, rs740406, rs7499892, rs7500448, rs7503807, rs751984, rs7525649, rs7560163, rs7568458, rs7617773, rs7633770, rs7678555, rs76954792, rs7696431, rs7770628, rs780094, rs7810507, rs7901016, rs7903146, rs7916879, rs7955901, rs7980458, rs7989336, rs80234489, rs8030379, rs8042271, rs806215, rs8090011, rs8108269, rs820429, rs838880, rs867186, rs871606, rs884366, rs885150, rs896854, rs897057, rs9266359, rs9268402, rs9299, rs9319428, rs9349379, rs9357121, rs9367716, rs9376090, rs9390698, rs944172, rs9470794, rs9473924, rs9505118, rs9534262, rs9552911, rs9568867, rs9593, rs9663362, rs9687065, rs975722, rs9810888, rs9815354, rs9818870, rs9828933, rs9892152, and rs9970807.

According to a specific embodiment of the present invention, in the present invention, said individual's information preferably further comprises one or more of BP, BMI, DM, TC, and Stroke-associated single-nucleotide polymorphism loci (preferably one or more groups, i.e. one or more of the BP group, the BMI group, the DM group, the TC group, and the Stroke group):
BP-related single nucleotide polymorphism loci: rs10051787, rs11651052, rs12037987, rs1275988, rs12999907, rs13041126, rs13143871, rs1558902, rs16896398, rs174546, rs17843768, rs1799945, rs391300, rs4336994, rs4722766, rs507666, rs6825911, rs7213603, rs7405452, rs880315, rs93138;
BMI-associated single nucleotide polymorphism loci: rs11257655, rs11604680, rs1470579, rs1982963, rs6545814, rs888789;
DM-associated single nucleotide polymorphism loci: rs10010670, rs10160804, rs1029420, rs1037814, rs1052053, rs10830963, rs10886471, rs10923931, rs11067763, rs11624704, rs11660468, rs117601636, rs1211166, rs12229654, rs12242953, rs12549902, rs12571751, rs1260326, rs12679556, rs12946454, rs13233731, rs13266634, rs13342232, rs1334576, rs1359790, rs1436953, rs1532085, rs1575972, rs16927668, rs16967013, rs17301514, rs17517928, rs17609940, rs17791513, rs17843797, rs1801282, rs1832007, rs2028299, rs2074158, rs2075423, rs2081687, rs2123536, rs2245019, rs2258287, rs2261181, rs2296172, rs2334499, rs243019, rs2487928, rs2642442, rs273909, rs2783963, rs2796441, rs2820315, rs2861568, rs2972146, rs3213545, rs340874, rs35879803, rs368123, rs3774472, rs3791679, rs3810291, rs3861086, rs3918226, rs3936511, rs4142995, rs42039, rs4275659, rs4458523, rs4757391, rs4765773, rs4846049, rs4923678, rs55783344, rs579459, rs58542926, rs6093446, rs634501, rs67156297, rs67839313, rs6825454, rs6831256, rs6871667, rs6878122, rs6909574, rs6984210, rs702634, rs7107784, rs7116641, rs7258189, rs7403531, rs748431, rs7528419, rs7610618, rs7616006, rs769449, rs78169666, rs7897379, rs7917772, rs79223353, rs79548680, rs820430, rs840616, rs9309245, rs9512699, rs9591012, rs984222;
TC-associated single nucleotide polymorphism loci: rs10401969, rs10889353, rs11136341, rs117711462, rs12027135, rs12453914, rs12927205, rs13115759, rs1367117, rs1495741, rs16844401, rs17122278, rs181359, rs2000813, rs2244608, rs2302593, rs247616, rs4883201, rs5996074, rs7134594, rs7258950, rs737337, rs7965082, rs964184;
Stroke-associated single nucleotide polymorphism loci: rs10203174, rs1050362, rs10947231, rs11634397, rs11957829, rs12500824, rs12607689, rs13702, rs1424233, rs1467605, rs1508798, rs16933812, rs17080091, rs17608766, rs180327, rs1878406, rs2075650, rs2107732, rs2237892, rs2295786, rs246600, rs2625967, rs2758607, rs2972143, rs34008534, rs35419456, rs376563, rs4471613, rs4724806, rs4777561, rs4939883, rs60154123, rs6544713, rs7136259, rs7193343, rs73596816, rs736699, rs7859727, rs7947761, rs832552.

According to a specific embodiment of the present invention, in the present invention, said individual's information preferably further comprises coronary artery disease clinical risk factors. In a specific embodiment of the present invention, said coronary artery disease clinical risk factors include: age, systolic blood pressure, total cholesterol, high density lipoprotein cholesterol, waist circumference, smoking, southern/northern populations, urban/rural populations, and family history of atherosclerotic cardiovascular diseases. In a specific embodiment, a China-PAR score may optionally be calculated based on the coronary artery disease clinical risk factors.

According to a specific embodiment of the present invention, in the present invention, a genetic risk score is obtained based on the information of the single nucleotide polymorphism loci by the following equation:

$$Genetic risk score = \sum β i \times Ni$$

wherein βi is an effect size of the i^{th} SNP, and Ni is the number of effect alleles of the i^{th} SNP carried by the individual.

According to a specific embodiment of the present invention, in the present invention, the effect sizes of the SNP are shown in Table 4.

According to a specific embodiment of the present invention, in the present invention, the higher the genetic risk score, the higher the individual's risk of developing coronary artery disease is. Said coronary artery disease includes myocardial infarction and/or angina pectoris.

According to a specific embodiment of the present invention, in the present invention, the individual to be tested is from an East Asian population, in particular a Chinese population.

In another aspect, the present invention also provides a device for evaluating a risk of developing coronary artery disease, comprising a detection unit and a data analysis unit, wherein:
said detection unit is used for obtaining information of an individual to be tested and providing detection results; wherein said information of the individual is the aforementioned individual's information; and
said data analysis unit is used for analyzing and processing the detection results from the detection unit.

According to a specific embodiment of the present invention, in the present invention, the analyzing and processing of the detection results from the detection unit by the data analysis unit comprises: assigning weighting factors to the detection results of said single nucleotide polymorphism loci to calculate a genetic risk score of said individual to be tested.

Preferably, said data analysis unit comprises:
a preprocessing module for normalizing the detection results of said single nucleotide polymorphism loci;
a calculation module for substituting the normalized detection results of the single nucleotide polymorphism loci into the following evaluation model to obtain a genetic risk score for the individual to be tested:

$$Genetic risk score = \sum β i \times Ni$$

wherein βi is an effect size of the i^{th} SNP, and Ni is the number of effect alleles of the i^{th} SNP carried by the individual.

According to a specific embodiment of the present invention, in the present invention, said data analysis unit further comprises a clinical factor processing module for obtaining a 10-year cardiovascular and cerebrovascular risk score by China-PAR of the individual to be tested.

According to a specific embodiment of the present invention, in the present invention, said calculation module is also used to further combine the genetic risk score with the clinical risk score to evaluate the 10-year incidence risk and/or lifetime risk information for coronary artery disease.

According to a specific embodiment of the present invention, in the present invention, said data analysis unit further comprises:
a matrix input module for receiving a plurality of the normalized detection results output by said preprocessing module and inputting the normalized detection results in a matrix form into the calculation module.

Preferably, said data analysis unit further comprises:
an output module for receiving the genetic risk score and/or the 10-year incidence risk and/or the lifetime risk information for coronary artery disease output from the calculation module, and outputting it as a diagnostic classification result.

In a specific embodiment of the present invention, the present invention integrates the genetic risk score with the clinical risk score of coronary artery disease, and establishs a simple risk evaluation chart (risk chart), which is easy to promote and use. Therefore, the data analysis unit of the device for evaluating the risk of developing coronary artery disease of the present invention may also include the risk evaluation chart (risk chart) of the present invention.

In yet another aspect, the present invention also provides a computer device comprising a memory, a processor, and a computer program stored in the memory and runnable on the processor, wherein when the processor executes said computer program, the device obtains an evaluation result of a risk of developing coronary artery disease of an individual based on information of the individual to be tested. Here, said individual's information is as previously described.

In another aspect, the present invention provides a method for evaluating the risk of developing coronary artery disease, the method comprising:
obtaining the information of the individual to be tested and providing detection results; wherein said individual's information is the aforementioned individual's information of the present invention; and
analyzing the detection results from the detection unit to evaluate the risk of developing coronary artery disease in the individual. The specific analysis process may be carried out in accordance with the aforementioned analysis process of the present invention.

In still another aspect, the present invention also provides a method of establishing a polygenic risk score for coronary artery disease, in particular a method of establishing a comprehensive polygenic risk score for coronary artery disease, the method comprising the steps of:
(1) screening SNPs to create a collection of single nucleotide polymorphism loci (SNPs) associated with coronary artery disease and/or coronary artery disease-related phenotypes; where the coronary artery disease-related phenotypes include: blood pressure, type 2 diabetes, blood lipids, obesity, and stroke;
(2) performing genotyping based on the single nucleotide polymorphism loci in step (1);
(3) extracting the risk alleles, effect sizes, and P values respectively of the measured SNPs corresponding to a plurality of subphenotypes from the results of a genome-wide association study and establishing a subphenotypic PRS for each subphenotype, said plurality of subphenotypes preferably including: coronary artery disease, body mass index, blood pressure, type 2 diabetes, total cholesterol, low density lipoprotein cholesterol, triglycerides, high density lipoprotein cholesterol, and stroke; preferably, wherein a plurality of candidate subphenotypic PRSs are established separately for each subphenotype and screened for the best subphenotypic PRS;
(4) determining the weights of each subphenotypic PRS;
(5) converting the weights of the subphenotypic PRS into weights at the SNP level;
(6) establishing a comprehensive polygenic risk score metaPRS for coronary artery disease.

According to specific embodiments of the present invention, in the method of establishing a polygenic risk score for coronary artery disease of the present invention, the coronary artery disease-associated phenotypic blood pressure includes: systolic blood pressure, diastolic blood pressure, pulse pressure, mean arterial blood pressure, and hypertension; the coronary artery disease-associated phenotypic obesity (body mass index) includes body weight index, waist circumference, and waist-to-hip ratio; and the coronary artery disease-associated phenotypic blood lipids includes total cholesterol, low density lipoprotein (LDL) cholesterol, triglycerides, and high density lipoprotein (HDL) cholesterol.

According to a specific embodiment of the present invention, in the method of establishing a polygenic risk score for coronary artery disease of the present invention, said plurality of subphenotypes include: coronary artery disease, body mass index, blood pressure, type 2 diabetes, total cholesterol, LDL cholesterol, triglycerides, HDL cholesterol, and stroke. That is, in the method of establishing a polygenic risk score for coronary artery disease of the present invention, the plurality of candidate subphenotypes PRSs established include: subphenotypes PRSs for coronary artery disease, stroke, type 2 diabetes, blood pressure, body mass index, total cholesterol, LDL cholesterol, triglycerides, and HDL cholesterol.

According to a specific embodiment of the present invention, in the method of establishing a polygenic risk score for coronary artery disease of the present invention, those that are found in genome-wide association studies to have genome-wide significant association with coronary artery disease or coronary artery disease-related phenotypes (coronary artery disease-related risk factors) are included in the collection of single nucleotide polymorphism loci. Specifically, in the collection of single nucleotide polymorphism loci are included: single nucleotide polymorphism loci associated with coronary artery disease, single nucleotide polymorphism loci associated with stroke, and single nucleotide polymorphism loci associated with blood pressure, type 2 diabetes, blood lipids, and obesity, respectively; and single nucleotide polymorphism loci associated with atherosclerosis clinical phenotypes may be further optionally incorporated. According to a specific embodiment of the present invention, in the method of establishing a coronary artery disease polygenic risk score of the present invention, said coronary artery disease polygenic risk score is used for evaluating the risk of developing coronary artery disease in an East Asian population; the single nucleotide polymorphism loci incorporated into the collection of single nucleotide polymorphism loci may be present in all populations, for example, those possibly including both European populations and East Asian populations, and the single nucleotide polymorphism loci associated with blood pressure, type 2 diabetes, blood lipids, obesity, and atherosclerosis clinical phenotypes may also be predominantly in East Asian populations.

According to a specific embodiment of the present invention, in the method for establishing a polygenic risk score for coronary artery disease of the present invention, a cohort population for the genotyping is an East Asian population.

According to a specific embodiment of the present invention, in the method of establishing a polygenic risk score for coronary artery disease of the present invention, the genotyping is performed using multiplex polymerase chain reaction targeted amplicon sequencing technology. The median sequencing depth is 982×.

According to a specific embodiment of the present invention, in the method of establishing a polygenic risk score for coronary artery disease of the present invention, SNPs with a genotype detection rate of less than 95% may be excluded from the genotyping process, and a collection of SNPs that are qualified for testing is obtained.

According to a specific embodiment of the present invention, in the method of establishing a polygenic risk score for coronary artery disease of the present invention, the risk alleles, effect sizes, and P-values of the measured SNPs corresponding to a plurality of subphenotypes are respectively extracted from the results of a large-scale genome-wide association study of an East Asian population. Here, preferably, the plurality of subphenotypes include: coronary artery disease, body mass index, blood pressure, type 2 diabetes, total cholesterol, low-density lipoprotein (LDL) cholesterol, triglycerides, high-density lipoprotein (HDL) cholesterol, and stroke. In the present invention, a subphenotype PRS is established separately for each subphenotype; preferably, multiple candidate subphenotypic PRSs are established separately for each subphenotype and the best subphenotypic PRS is selected. More specifically, N groups of SNPs can be set up according to the extracted P values (preferably pruned according to a linkage disequilibrium of r²<0.2), N being greater than or equal to 2, and N candidate subphenotypic PRSs can be established for each subphenotype and the best subphenotypic PRS can be selected.

According to a specific embodiment of the present invention, in the method for establishing a polygenic risk score for coronary artery disease of the present invention, the process of establishing a PRS for each subphenotype comprises:
setting up multiple SNP groups on the basis of the extracted P-values, and for each group of SNPs, pruning according to r²<0.2 based on the cohort population data using the clumping command of the PLINK software to obtain multiple SNP combinations;
using genotype data, weighting and summing up the number of SNP risk alleles (0, 1, or 2) according to their corresponding effect sizes to establish a plurality of candidate PRSs incorporating different SNP combinations, evaluating the correlation of these candidate PRSs with coronary artery disease using a logistic regression modeling, and selecting the score with the largest odds ratio (OR) (for an increment of one standard deviation in PRS) as the best subphenotypic PRS.

According to a more specific embodiment of the present invention, in the above process of establishing a PBS for each subphenotype, N groups of SNPs may be set up according to the extracted P-values, N being greater than or equal to 2. For example, 9, 10, 11 or 12 groups may be selected according to P-values of 0.5, 0.4, 0.3, 0.2, 0.1, 0.05, 0.01, 10⁻³, 10⁻⁴, 10⁻⁵, 10⁻⁶, 10⁻⁷.

According to a more specific embodiment of the present invention, in the above process of establishing a PBS for each subphenotype, when N groups of SNPs are set up according to the extracted P-values according to a linkage disequilibrium of r²<0.2, N groups of SNPs can be obtained, that is, N candidate PRSs incorporating different combinations of SNPs can be established.

In the present invention, the correlation coefficient r and P-values between every two of the subphenotypic PRSs may be further calculated by Pearson correlation analysis.

According to a specific embodiment of the present invention, in the method for establishing a polygenic risk score for coronary artery disease of the present invention, a portion of the population may be selected from all in the cohort population in a predetermined proportion as a training set (the remaining portion of the population may be used as a validation set). The processes of establishing subphenotypic PRSs and determining the weights of each subphenotypic PRS may be performed independently in the training set, respectively.

According to a specific embodiment of the present invention, in the method of establishing a polygenic risk score for coronary artery disease of the present invention, the process of determining the weights of each subphenotypic PRS comprises:
converting each subphenotypic PRS into normalized scores with a mean of 0 and a standard deviation of 1;
using a training set, putting each of the normalized subphenotypic PRSs and the covariates to be adjusted (age, sex) together into an elastic net logistic regression model, and selecting the model with the highest AUC as the final model from which the coefficients of each PRS (β1 . . . βₙ, a total of n PRSs) are obtained as weights.

In some specific embodiments of the present invention, the elastic net logistic regression model may correct the correlation among the individual subphenotypic PRSs. This model is used in the present invention to evaluate the association of 9 (i.e., n is 9) subphenotypic PRSs with coronary artery disease, and compare and analyze the ORs of the elastic net logistic regression estimation with those of a univariate logistic regression estimation. Further, the present invention establishes and validates a metaPRS for coronary artery disease by integrating the 9 subphenotypic PRSs and converting the weights of the subphenotypic PRSs into weights at the SNP level.

According to specific embodiments of the present invention, in the method of establishing a polygenic risk score for coronary artery disease of the present invention, the process of converting the weights of the subphenotypic PRS into weights at the SNP level is performed according to the following model:

$$βsnp\_i = \frac{{β}_{1}}{{σ}_{1}} {α}_{j 1} + … + \frac{{β}_{n}}{{σ}_{n}} {α}_{jn}$$

wherein, σ₁ . . . , σᵢ is the standard deviation of each subphenotypic PRS (a total of n) in the training set, and αⱼ₁, . . . , αⱼₙ is the effect size of the i^{th} SNP corresponding to each subphenotype, and if a SNP is not included in the k^{th} score, the effect value αⱼₖ of that SNP is set to 0.

According to a specific embodiment of the present invention, in the method of establishing a polygenic risk score for coronary artery disease of the present invention, after the weights of the subphenotypic PRSs are converted into weights at the SNP level, a comprehensive score metaPRS for polygenic genetic risk of coronary artery disease is further established with the weights at the SNP level:

$$metaPRS = \sum βsnp\_i \times Ni$$

wherein, βsnp_i is the effect size of the i^{th} SNP, and Ni refers to the number of effect alleles of the i^{th} SNP carried by the individual.

According to a specific embodiment of the present invention, the method of establishing a comprehensive polygenic risk score for coronary artery disease of the present invention may further comprise a process of evaluating the function of the established metaPRS in the prediction and stratification of the risk of coronary artery disease.

According to specific embodiments of the present invention, in the method of establishing a polygenic risk score for coronary artery disease of the present invention, preferably, by using the 20th and 80th percentiles of the metaPRS of all individuals in the cohort population as cut-offs, the individual is categorized into a population having a low, medium, or high risk of genetic incidence of coronary artery disease.

In another aspect, the present invention also provides a device for establishing a comprehensive polygenic risk score for coronary artery disease, the device comprising:
a genotyping module for genotyping;
a subphenotype PRS establishment module for extracting the risk alleles, effect sizes, and P values respectively of the measured SNPs corresponding to a plurality of subphenotypes from the results of a genome-wide association study and establishing a subphenotypic PRS for each subphenotype; preferably, wherein a plurality of candidate subphenotypic PRSs is established for each subphenotype and the best subphenotypic PRS is selected;
a model training module for determining the weights of each subphenotypic PRS in a training set; and
a metaPRS establishment module for converting the weights of the subphenotypic PRS into weights at the SNP level and establishing a comprehensive polygenic risk score (metaPRS) for coronary artery disease.

According to a specific embodiment of the present invention, the device for establishing a comprehensive polygenic risk score for coronary artery disease of the present invention further optionally includes an SNP screening module, which is used for screening a collection of single nucleotide polymorphism loci (SNPs) associated with coronary artery disease or a coronary artery disease-related phenotype.

According to a specific embodiment of the present invention, the genotyping module in the device for establishing a comprehensive polygenic risk score for coronary artery disease of the present invention may also be used to exclude SNPs with a genotype detection rate of less than 95% after genotyping.

According to a specific embodiment of the present invention, in the device for establishing a comprehensive polygenic risk score for coronary artery disease of the present invention, optionally, the metaPRS establishment module may be further used for evaluating the function of the established metaPRS in the prediction and stratification of the risk of coronary artery disease.

In yet another aspect, the present invention also provides a computer device comprising a memory, a processor and a computer program stored on the memory and runnable on the processor, wherein when the processor executes the computer program, the device evaluates the risk of developing coronary artery disease in an individual by using a comprehensive coronary artery disease polygenic risk score established by the method described in the present invention.

In some specific embodiments of the present invention, a genome-wide association study has been conducted in 51,531 patients with coronary artery disease and 215,934 patients without coronary artery disease. Genetic information on nine phenotypes of coronary artery disease and associated phenotypes were then integrated to establish polygenic risk scores in 2,800 coronary artery disease cases and 2,055 healthy controls, and finally validated and evaluated in a prospective cohort of 41,271 cases in a Chinese population. The established polygenic risk scores were found to have excellent predictive value for the incidence of coronary artery disease. Individuals in different genetic risk groups showed different pathogenesis. With an increment of one standard deviation in metaPRS, the relative risk of developing coronary artery disease is increased by 44%. Grouped by tertiles (<20%, 20% to 80%, >80%), the risk of developing coronary artery disease in individuals with a high genetic risk (>80%) was three times higher than that in individuals with a low genetic risk (<20%), and the cumulative risk of coronary artery disease before the age of 80 in both groups was 5.8% and 16.0%, respectively.

Also, the results of the present invention show that the polygenic genetic scores can further refine the risk stratification for coronary artery disease development on the basis of a clinical risk. In particular, a genetic risk can be used to re-stratify individuals at medium and high clinical risks to a considerable extent. For example, in the high clinical risk group, the relative risk of coronary artery disease in those with a high genetic risk was 3.82 times higher than those with a low genetic risk (HR: 3.82; 95% CI: 2.70-5.41), and there was also a 3.8-fold difference in the 10-year cumulative incidence rate of coronary artery disease (10-year cumulative incidence of coronary artery disease in the low- and high-genetic-risk groups was 2.0% and 7.6%, respectively). That is, in the cohort of the present invention, 20% of the 6,768 individuals identified to have a high risk by the China-PAR rating could be reclassified to a medium risk upon the genetic risk evaluation. In contrast, among the 8,342 individuals with a medium clinical risk identified by the China-PAR rating, those with a genetic risk within the 80%-100% quartile had a corresponding absolute risk of coronary artery disease (a 10-year risk of 3.8%, and a lifetime risk of 16.9%) that reached the level of a population with a high clinical risk and a medium genetic risk (a 10-year risk of 4.0%, and a lifetime risk of 17.4%). As age is the most important driving factor in the clinical risk score, it is overrated for the risk in the elderly, and early-onset coronary artery disease cases are also underdiagnosed. Meanwhile, genetic risks are independent of age and can be determined early in life and before the emergence of clinical risk factors.

The studies in the present invention demonstrate that the polygenic genetic scores in combination with traditional clinical risk scores has important application prospects for refining and re-stratifying the risk of developing coronary artery disease.

### BRIEF DESCRIPTION OF THE ACCOMPANYING FIGURES

FIG. 1 shows a flowchart of the study of the present invention; here, PRS, polygenic risk score.

FIG. 2 shows the association between coronary artery disease PRSs and coronary artery disease in a training set compared using East Asian and European American GWAS effect sizes. Logistic regression models were used to calculate odds ratios (ORs) and 95% confidence intervals (CIs), adjusted for age and sex. Scores were calculated using effect sizes from the East Asian population and European UK Biobank coronary artery disease GWAS data as the weights of SNPs, respectively. Different P-value thresholds were set (0.5, 0.4, 0.3, 0.2, 0.1, 0.05, 0.01, 10⁻³, 10⁻⁴, 10⁻⁵, 10⁻⁶, 10⁻⁷) to establish 12 PRSs containing different combinations of SNPs, respectively (linkage disequilibrium r²<0.2).

FIG. 3 shows the association of subphenotypic PRSs (per standard deviation increment) with CAD in the training set at different P-value thresholds. Logistic regression was used to calculate the ratio of ratios (OR) and 95% confidence intervals (CI), adjusted for age and sex.

FIG. 4, PRS correlation plots for each subphenotype, wherein *P<0.05, **P<10⁻³, ***P<10⁻¹⁰.

FIG. 5 shows the association between subphenotypic PRSs (per standard deviation increment) and coronary artery disease in the training set. Odds ratios (OR) and 95% confidence intervals (CI) were calculated using logistic regression and elastic net logistic regression respectively, adjusted for age and sex.

FIG. 6 shows the hazard ratios of metaPRS (per standard deviation increment) and subphenotypic PRS to CAD onset in the prospective cohort. A Cox model was used for analysis, using age as the time scale and adjusted for cohort origin and sex.

FIG. 7 shows the relative and absolute risks of developing coronary artery disease for different genetic groups (groups of <20%, 20%-80%, and >80%). Here, Cox models were used to estimate the HR and 95% CI and cumulative incidence of coronary artery disease in different genetic risk groups, adjusted for sex and cohort origin, scaled by age, and accounted for competing risks. Dashed lines indicate 95% CI. CAD, coronary artery disease; HR, hazard ratio; CI, confidence interval.

FIG. 8 shows the relative and absolute risks of developing coronary artery disease in different genetic groups (groups of <20%, 20%-80%, and >80%) stratified by sex. In this, Cox models were used to estimate the HR and 95% CI and cumulative incidence of coronary artery disease in different genetic risk groups, adjusted for sex and cohort origin, scaled by age, and accounted for competing risks. Dashed lines indicate 95% CI. CAD, coronary artery disease; HR, hazard ratio; CI, confidence interval.

FIG. 9 shows the relative and absolute risks of coronary artery disease grouped according to family history of coronary artery disease and genetic risk score. Cox proportional risk models accounted for competing risks were used to estimate the HRs and 95% CIs as well as cumulative risk of coronary artery disease, using age as the time scale and adjusted for sex and cohort.

FIG. 10 shows the 10-year and lifetime risks of developing coronary artery disease for the three genetic risk groups at different clinical risks. a. The 10-year risk of coronary artery disease incidence was obtained using a Cox proportional risk model with years of follow-up as the time scale and adjusted for sex and cohort. b. The lifetime risk of coronary artery disease (up to the age of 80) was obtained using a proportional regression model of competing risks, which took into account competing risks with age as the time scale. risk and adjusted for sex and cohort.

FIG. 11 shows the relative and absolute risks of coronary artery disease incidence in three genetic risk groups with different clinical risks. Sex-, age-, and cohort-adjusted Cox proportional risk models were used to estimate the hazard ratio (95% confidence intervals) and cumulative risk of coronary artery disease.

FIG. 12 shows a chart for evaluating a 10-year risk for developing coronary artery disease that combines clinical risk scores and genetic scores. The absolute 10-year risk of coronary artery disease in different age and sex groups was calculated using the Cox proportional risk model, with polygenic risk scores grouped according to quintiles and clinical risk grouped according to 10-year risk scores for atherosclerotic cardiovascular diseases of <5%, 5-9.9%, 10-14.9%, or ≥15%.

FIG. 13 shows a chart for evaluating a lifetime risk of coronary artery disease grouped according to clinical risks and genetic risks. The lifetime risk of coronary artery disease (up to the age of 80) for different age and sex populations was modeled using a proportional risk model that takes into account of competing risks, with polygenic risk scores grouped according to quintiles, and clinical risk grouped according to 10-year risk scores for atherosclerotic cardiovascular diseases of <5%, 5-9.9%, 10-14.9%, or ≥15%.

FIG. 14 shows the distribution of genetic risk scores across the population for an individual to be tested in a specific example.

### DETAILED DESCRIPTION OF THE INVENTION

In order to have a clearer understanding of the technical features, objects and beneficial effects of the present invention, the technical solutions of the present invention are described in detail below in conjunction with specific embodiments and the accompanying drawings, and it should be understood that these examples are used only to illustrate the present invention and are not intended to limit the scope of the present invention. To a person skilled in the art, various changes and/or modifications readily contemplated within the spirit of the present invention, such as partial additions, deletions and/or substitutions on the basis of a plurality of SNP collections identified in the present invention without substantively affecting the results of the assessment, are all recognized as being covered within the scope of protection of the present invention. In the Examples, each of the starting reagents and materials is commercially available, and the experimental methods for which specific conditions are not indicated are conventional processes and conditions well known in the related field, or as recommended by the instrument manufacturer.

### Example 1

### Designed Procedure and Population of the Study

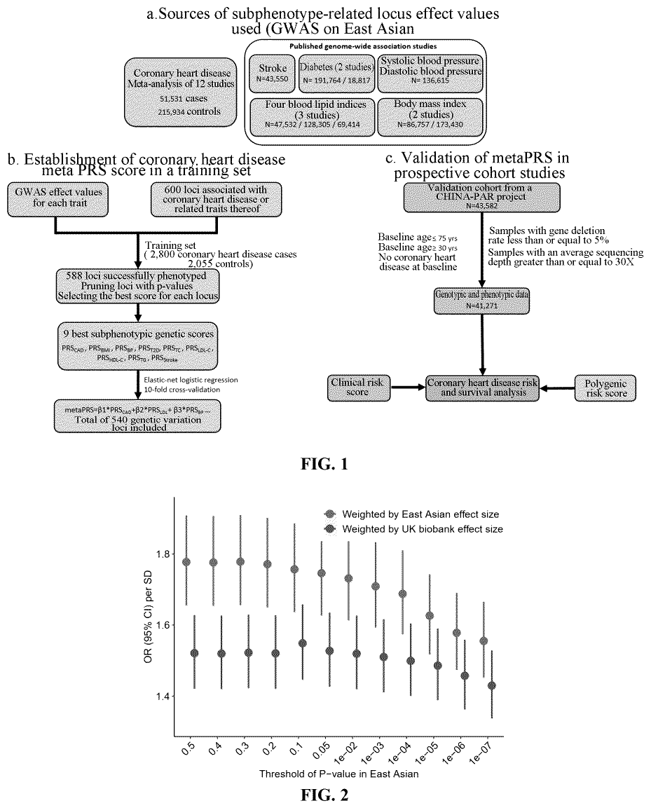

The study design flowchart is shown in FIG. 1. The present invention developed a polygenic risk score (PRS) for CAD in 2,800 CAD patients and 2,055 healthy controls (Table 1), and then validated it in a large prospective cohort population. The CAD cases in the training set were from Fu Wai Hospital, Chinese Academy of Medical Sciences. The diagnosis of myocardial infarction (MI) strictly follows diagnostic criteria based on signs, symptoms, electrocardiogram and cardiac enzyme activities. Coronary artery disease was diagnosed in conjunction with the presence or absence of a previous diagnosis of myocardial infarction, or a stenosis of more than 50% in the main left coronary artery, or a stenosis of >70% in at least one major epicardial vessel.

The validation cohort was drawn from three sub-cohorts of China-PAR studies, including the China Multicenter Collaborative Study on Cardiovascular Health (InterASIA), the China Multicenter Collaborative Study on Cardiovascular Epidemiology (ChinaMUCA-1998), and the China Intervention for Metabolic Syndrome in Communities and Family Health in China (CIMIC) study (Yang, X. et al. Predicting the 10-Year Risks of Atherosclerotic Cardiovascular Disease in Chinese Population: The China-PAR Project (Prediction for ASCVD Risk in China). Circulation 134, 1430-1440 (2016)). Briefly, the ChinaMUCA-1998, InterASIA, and CIMIC baselines were established in 1998, 2000-2001, and 2007-2008, respectively. According to uniform criteria, the first follow-ups of the InterASIA and ChinaMUCA-1998 were conducted in 2007-2008, and all three cohorts were followed up uniformly in 2012-2015 and 2018-2020. In this study, blood samples and data on key covariates were collected from a total of 43,582 participants independent of the training set. A total of 41,271 participants were ultimately included in the analysis after exclusion of 561 individuals with high genotypic deletion rates (>5.0%) or low mean sequencing depth (<30×), 1,352 individuals who were <30 or >75 years old at baseline, and 398 individuals with confirmed coronary artery disease at baseline.

All studies were approved by the Ethical Review Committee of Fu Wai Hospital, Chinese Academy of Medical Sciences. Each participant had signed an informed consent form before data collection.

TABLE 1
General information of the training set

| Characteristics | Controls | | Cases | |
|---|---|---|---|---|
| Sample size, N | 2055 | | 2800 | |
| Males (%) | 58.5 | | 69.3 | |
| Baseline age of cohort, year | 54.77 | (7.53) | — | |
| Age of onset, year | — | | 51.59 | (7.36) |
| Body mass index, kg/m² | 25.05 | (3.29) | 26.12 | (3.71) |
| Total cholesterol, mg/dl | 193.1 | (34.3) | 170.19 | (45.61) |
| LDL cholesterol, mg/dl | 112.75 | (29.82) | 98.14 | (39.03) |
| HDL cholesterol, mg/dl | 52.31 | (12.33) | 41.47 | (11.25) |
| Triglycerides, mg/dl | 147.19 | (111.26) | 168.92 | (118.03) |
| Systolic blood | 132.35 | (17.88) | 121.68 | (16.55) |
| pressure, mmHg | | | | |
| Diastolic blood | 83.32 | (10.91) | 76.53 | (11.33) |
| pressure, mmHg | | | | |

| | | |
|---|---|---|
| Hypertension (%) | 38.9 | 39.1 |
| Smoking (%) | 47.3 | 63.3 |
| Alcohol consumption (%) | 45.6 | 49.9 |

Values in mean (SD) or N (%).

### Data Collection and Definition of Risk Factors

Essential information was collected at baseline and during follow-ups by trained investigators under strict quality control. A normalized questionnaire was used to collect personal information (gender, date of birth, etc.), lifestyle information (dietary habits, physical activities, etc.), history of diseases and family history of CAD. Participants also underwent a physical examination (weight, height, blood pressure, etc.) and provided a fasting blood sample to measure blood lipid and glucose levels.

To obtain disease outcome and death-related information during follow-ups, researchers followed up with participants or their proxies and also collected the participants' medical records (or death certificates). Two committee members independently verified the outcome events. If there were inconsistencies, other committee members would step in to discuss until a consensus was eventually reached. Coronary artery disease onset was defined as the first occurrence of unstable angina, nonfatal acute myocardial infarction, or the occurrence of coronary artery disease death. A fatal event caused by myocardial infarction or other coronary artery diseases was defined as a coronary artery disease death. The time interval between the baseline date and the date of onset of coronary artery disease, the date of death, or the date of the last follow-up visit was the years of follow-up.

The present invention defines the following coronary artery disease risk factors: dyslipidemia, hypertension, diabetes, BMI, smoking, and family history of coronary artery disease. Dyslipidemia is defined as TC ≥240 mg/dl and/or LDL-C ≥160 mg/dl and/or TG ≥200 mg/dl and/or HDL-C <40 mg/dl and/or administration of lipid-lowering medication within the past 2 weeks. Hypertension was defined as systolic blood pressure ≥140 mmhg and/or diastolic blood pressure ≥90 mmhg and/or administration of antihypertensive medication within the past 2 weeks. Diabetes was defined as fasting blood glucose level ≥126 mg/dl and/or administration of insulin and/or oral hypoglycemic medication and/or having a history of diabetes. BMI was calculated as weight (kg) divided by squared height (m). Smoking was determined by self-reported smoking status of the study subjects. For family history of coronary artery disease, the invention considered the incidence of CAD in any first-degree relatives (father, mother, or siblings).

### Genetic Variation Loci Selection and Genotyping

The present invention began with a selection of 600 genetic variant loci that had been found to have genome-wide significant association (P<5×10⁻⁸) with coronary artery disease (n=212) or coronary artery disease-associated risk factors in genome-wide association studies, including stroke (n=42), blood pressure (n=56), blood lipids (n=130), T2D (n=90), and obesity (n=79) (Table 2). Information on all genetic variant loci has been provided in Table 3. In short, for coronary artery disease, the present invention selected all the genetic loci reported in East Asian and European populations; for other risk factors, the present invention focused on the genetic loci reported in East Asian populations.

Training set samples were genotyped using a Multi-Ethnic Genotyping Array (MEGA) chip from Infinium to obtain genetic variant information at the tested loci. In the cohort population, the present invention used multiplex PCR targeted amplicon sequencing to genotype the samples. Multiplex primers were designed for each mutation using conventional procedures in the art, and the amplicon target regions were high-throughput sequenced using an Illumina Hiseq X Ten sequencer. After excluding 12 variants with a detection rate of <95% or missing in the training dataset, a total of 588 variants or their substitutions were successfully detected, with an average detection rate of 99.9% and a median sequencing depth of 982×. To evaluate the reproducibility of genotyping, 1,648 samples was genotyped multiple times in the present invention, with a >99.4% consistency of the identification results.

TABLE 2
Sources of genetic variants selected in this study

| | No. of | |
|---|---|---|
| Traits | variants | Reference |
| CAD | 212 | Lu et al. Nikpay et al. Nelson et al. |
| | | Howson et al. Klarin et al. van de et al. |
| | | Deloukas et al. Verweij et al. |
| BP (SBP, DBP, PP, MAP, HTN) | 56 | Lu et al. Kato et al. |
| T2D | 90 | Imamura et al. |
| Obesity (BMI, WC, WHR) | 79 | Wen et al. Wen et al. |
| Lipid (TC, LDL-C. TG, HDL-C) | 130 | Lu et al. Lu et al. Spracklen et al. |
| Stroke | 42 | Traylor et al. Lee et al. Chauhan et al. |
| | | Cheng et al. Woo et al. Malik et al. Carty |
| | | et al. SiGN et al. Gudbjartsson et |
| | | al. Gretarsdottir et al. Holliday et al. |

| | |
|---|---|
| Total | 600 |

CAD, coronary artery disease; SBP, systolic blood pressure; DBP, diastolic blood pressure; PP, pulse pressure; MAP, mean arterial pressure; HTN, hypertension; T2D, type 2 diabetes; BMI, body mass index; WC, waist circumference; WHR, waist-to-hip ratio; TC, total cholesterol; LDL-C, low-density lipoprotein cholesterol; TG, triglycerides; HDL-C, high-density lipoprotein cholesterol.

Establishment of metaPRS
(1) Extraction of SNP Effect Sizes from GWAS Result Data and Calculation for Each Subphenotype PRS

The present invention first established genetic scores for nine CAD-associated phenotypes based on effect sizes from large-scale genome-wide association studies in an East Asian population. To accurately estimate the CAD effect sizes of the selected variants in the East Asian population, a genome-wide association study of coronary artery disease in an East Asian population with a total sample size of 267,465 cases (51,531 patients with coronary artery disease and 215,934 patients without coronary artery disease) was conducted in the present invention. For the other 8 phenotypes (stroke, type 2 diabetes, blood pressure, body mass index, total cholesterol, low-density lipoprotein cholesterol, triglycerides, and high-density lipoprotein cholesterol), the present invention obtained risk alleles, effect sizes, and P values corresponding to each subphenotype for each locus from large genome-wide association studies published on East Asian populations. A detailed list of the selected studies is shown in Table 3.

TABLE 3
Summary of data sources used for polygenic risk score calculation

| | | | | Sample size | | |
|---|---|---|---|---|---|---|
| | | | | (case/ | | |
| Trait | Source | Types | Ancestry | control) | Method | Reference |
| CAD | BAS | GWAS | Chinese | 505/ | Meta- | Lu et al. |
| | | | | 1,021 | analysis | |
| | CAS | GWAS | Chinese | 1,010/ | | Lu et al. |
| | | | | 3,998 | | |
| | BBJ | GWAS | Japanese | 29,319/ | | Koyama et al. |
| | | | | 183,134 | | |
| | FWBB | Panel | Chinese | 9,223/ | | — |
| | | | | 5,160 | | |
| | HuCAD | EWAS | Chinese | 4,664/ | | Zhang et al. Wang |
| | | | | 4,533 | | et al. |
| | PUUMA | EWAS | Chinese | 1,463/ | | Tang et al. |
| | | | | 5,987 | | |
| | HKU-TRS | EWAS | Chinese | 2,372/ | | Tang et al. |
| | | | | 3,388 | | |
| | SCHS | GWAS | Chinese | 718/ | | Han et al. |
| | | | | 1,262 | | |
| | SCES | GWAS | Chinese | 631/ | | Han et al. |
| | | | | 1,713 | | |
| | SP2 | GWAS | Chinese | 429/ | | Han et al. |
| | | | | 2,189 | | |
| | SIMES | GWAS | Malaysian | 391/ | | Han et al. |
| | | | | 2,212 | | |
| | CAGE | GWAS | Japanese | 806/ | | Takeuchi et al. |
| | | | | 1,337 | | |
| | Total | | | 51,531/ | | |
| | | | | 215,934 | | |
| BP | BBJ | GWAS | Japanese | 136,615 | Average of | Kanai et al. |
| | | | | | systolic and | |
| | | | | | diastolic | |
| | | | | | blood | |
| | | | | | pressure | |
| T2D | BBJ | GWAS | Japanese | 36,614/ | Meta- | Suzuki et al. |
| | | | | 155,150 | analysis | |
| | AGEN | GWAS | East Asian | 6,952/ | | Yoon et al. |
| | | | | 11,865 | | |
| BMI | BBJ | GWAS | Japanese | 173,430 | Minimum P- | Akiyama et al. |
| | AGEN | GWAS | East Asian | 86,757 | value | Wen et al. |
| Lipid | Meta-analysis | EWAS | East Asian | 47,532 | Minimum P- | Lu et al. |
| (LDL-C, | BBJ | GWAS | Japanese | 128,305 | value | Kanai et al. |
| HDL-C, | AGEN | GWAS | East Asian | 69,414 | | N. Spracklen et al. |
| TC, TG) | | | | | | |
| Stroke | BBJ | GWAS | Japanese | 16,256/ | Original | Malik et al. |
| | | | | 27,294 | values | |

GWAS, genome-wide association study; EWAS, exome-wide association study; BP, blood pressure; CAD, coronary artery disease; T2D, type 2 diabetes; BMI, body mass index; TC, total cholesterol; LDL-C, low-density lipoprotein cholesterol; TG, triglycerides; HDL-C, high-density lipoprotein cholesterol.

Taking subphenotypic CAD as an example, the present invention integrated large-scale coronary artery disease case-control genomic data from East Asian and Chinese populations to conduct a genome-wide association study of coronary artery disease, with samples of up to 51,531 patients with coronary artery disease and 215,934 patients with no coronary artery disease, and Meta-analysis was done on the results of the association analysis of the different sub-cohorts using a fixed-effects model, to obtain the risk alleles, effect sizes and P values of the measured SNPs. Based on the extracted P values, 12 groups of SNIPs were screened according to 0.5, 0.4, 0.3, 0.2, 0.1, 0.05, 0.01, 10⁻³, 10⁻⁴, 10⁻⁵, 10⁻⁶, 10⁻⁷, and for each group of SNIPs, based on the data of the cohort population, they were pruned according to a linkage disequilibrium of r²<0.2 using the clumping command of the PLINK software (version 1.9). Twelve sets of SNIP combinations were finally obtained. Using the training set genotype data, the number of individual SNIP risk alleles (0, 1, or 2) was weighted and summed according to their corresponding effect sizes to establish 12 candidate PRSs incorporating different combinations of SNIPs, and a logistic regression model was used to evaluate the association between these candidate PRSs and coronary artery disease, and the scores with the largest odds ratios (ORs) (for an increment of one standard deviation in PRS) were selected as the best PRS for coronary artery disease. For the other 8 phenotypes, SNP effect sizes were obtained from literatures as provided in Table 3 for the corresponding phenotypes, and the other 8 subphenotypic PRSs were then established by following the same steps as described above. Among them, the SNP loci utilized by the best subphenotypic PRS and the effect sizes are shown in Table 4.

### (2) Calculation of the Weights of Each Subphenotypic PRS in the Training Set

The 9 subphenotypic PRSs were converted into scores with a mean of 0 and a standard deviation of 1. Using the training set, the normalized 9 subphenotypic PRSs and the covariates to be adjusted (age, gender) were jointly placed into a elastic net logistic regression model (cv.glmnet function, R package “glmnet”), in which a range of different penalty items (set alpha=0, 0.1, 0.2, 0.3, 0.4, 0.5, 0.6, 0.7, 0.8, 0.9, or 1.0) were evaluated using 10-fold cross-validation, and the model parameter type.measure is set to “auc”. The model with the highest AUC (area under receiving-operator characteristic curve) was automatically chosen as the final model, and the coefficients of each PRS (β1 . . . β9) were obtained as weights. The weights of each subphenotype PRS are provided in Table 5, and the subphenotypes TG, HDL, and LDL were given a weight of zero.

### (3) Conversion of Subphenotypic PRS Weights to SNP Level Weights

$$βsnp\_i = \frac{{β}_{1}}{{σ}_{1}} {α}_{j 1} + … + \frac{{β}_{9}}{{σ}_{9}} {α}_{j 9}$$

The weights at the PRS level were converted to weights at the SNP level using the above equation, where σ₁, . . . , σᵢ is the standard deviation of each subphenotypic PRS in the training set, and αⱼ₁, . . . , αⱼₙ is the effect size of the i^{th} SNP corresponding to each subphenotype, and if a certain SNP is not included in the k^{th} score, the effect sizeαⱼₖ of that SNP is set to 0.
(4) Calculation of metaPRS
MetaPRS of an individual was calculated using the formula: metaPRS=Σβsnp_i×Ni, where βsnp_i is the effect size of the i^{th} SNP (i.e., the weight at the SNP level obtained in Step 3), and Ni is the number of effect alleles of the i^{th} SNP carried by the individual.
After the statistical processing step, a total of 510 SNPs having a non-zero weight were finally obtained and included in the calculation of metaPRS, and the information and weights of all eligible SNPs are provided in Table 4.
(5) metaPRS Cut-Offs
The 20% and 80% percentiles of the metaPRS for all individuals in the cohort population were used as cut-offs to classify individuals as being at low, medium, or high genetic risk for coronary artery disease.

TABLE 4
Information and weights of SNPs identified in the present invention

| | Subphenotypic PRS | | | | metaPRS | | |
|---|---|---|---|---|---|---|---|
| | Sub- | Effect | Other | SNP effect | Effect | Other | SNP effect |
| SNP | phenotype | allele | allele | size | allele | allele | size |
| rs10064156 | CAD | T | C | −0.0132 | C | T | 0.00822464 |
| rs10071096 | CAD | A | G | −0.0566 | A | G | −0.05222655 |
| rs10093110 | CAD | A | G | −0.0177 | A | G | −0.01742527 |
| rs10096633 | CAD | T | C | −0.0852 | T | C | −0.07637729 |
| rs10139550 | CAD | C | G | −0.0394 | G | C | 0.03684048 |
| rs10237377 | CAD | T | G | −0.0332 | G | T | 0.02865684 |
| rs10260816 | CAD | C | G | −0.0263 | C | G | −0.01814896 |
| rs10267593 | CAD | A | G | −0.0437 | A | G | −0.03877288 |
| rs1027087 | CAD | A | T | −0.0138 | T | A | 0.01131546 |
| rs10278336 | CAD | A | G | 0.0192 | G | A | −0.02244779 |
| rs10455782 | CAD | T | C | 0.0138 | T | C | 0.01273368 |
| rs10503675 | CAD | A | G | −0.0138 | G | A | 0.01082104 |
| rs10512861 | CAD | T | G | −0.0289 | T | G | −0.02869826 |
| rs10513801 | CAD | T | G | 0.0621 | G | T | −0.0690893 |
| rs10745332 | CAD | A | G | 0.0213 | G | A | −0.03132162 |
| rs10757274 | CAD | A | G | −0.1836 | G | A | 0.17118285 |
| rs10773003 | CAD | A | G | −0.0264 | A | G | −0.02436009 |
| rs10842992 | CAD | T | C | 0.0134 | C | T | −0.02164156 |
| rs10846744 | CAD | C | G | 0.0266 | G | C | −0.02832959 |
| rs10857147 | CAD | A | T | −0.053 | T | A | 0.08300079 |
| rs10890238 | CAD | A | T | −0.0546 | T | A | 0.05064862 |
| rs10953541 | CAD | T | C | −0.0254 | T | C | −0.02391389 |
| rs10968576 | CAD | A | G | −0.0176 | G | A | 0.01624006 |
| rs11030104 | CAD | A | G | 0.0376 | G | A | −0.06956572 |
| rs11057830 | CAD | A | G | 0.0261 | A | G | 0.02267324 |
| rs11067762 | CAD | A | G | −0.0336 | A | G | −0.04332208 |
| rs11077501 | CAD | T | C | −0.0181 | C | T | 0.01630849 |
| rs11099493 | CAD | A | G | 0.0418 | G | A | −0.03872062 |
| rs11107829 | CAD | A | C | 0.0857 | C | A | −0.07907801 |
| rs11125936 | CAD | T | C | 0.0343 | C | T | −0.03699814 |
| rs11142387 | CAD | A | C | −0.0157 | C | A | 0.01675164 |
| rs1116357 | CAD | A | G | −0.0096 | G | A | 0.01329774 |
| rs11170820 | CAD | C | G | −0.1038 | G | C | 0.09329318 |
| rs11205760 | CAD | T | C | 0.0198 | C | T | −0.0268054 |
| rs11206510 | CAD | T | C | 0.0371 | C | T | −0.04171447 |
| rs11509880 | CAD | A | G | 0.0129 | G | A | −0.01460955 |
| rs11556924 | CAD | T | C | −0.1171 | T | C | −0.10894629 |
| rs11557092 | CAD | T | C | −0.079 | T | C | −0.07327192 |
| rs115696548 | CAD | T | C | −0.066 | C | T | 0.06435618 |
| rs11601507 | CAD | A | C | 0.0589 | A | C | 0.0663222 |
| rs11677932 | CAD | A | G | −0.0114 | A | G | −0.00940288 |
| rs1169288 | CAD | A | C | −0.0205 | C | A | 0.01891598 |
| rs1173766 | CAD | T | C | −0.021 | T | C | −0.03512404 |
| rs11787792 | CAD | A | G | 0.0469 | G | A | −0.06021268 |
| rs11810571 | CAD | C | G | −0.0217 | C | G | −0.02342924 |
| rs11838267 | CAD | T | C | 0.0402 | C | T | −0.03518112 |
| rs11838776 | CAD | A | G | 0.0747 | A | G | 0.0831271 |
| rs11847697 | CAD | T | C | −0.5391 | T | C | −0.49744404 |
| rs11911017 | CAD | T | G | 0.0115 | T | G | 0.01040239 |
| rs12175867 | CAD | T | C | 0.0271 | C | T | −0.02523172 |
| rs12214416 | CAD | A | T | 0.1923 | A | T | 0.18699491 |
| rs12445022 | CAD | A | G | −0.0223 | A | G | −0.01990807 |
| rs12463617 | CAD | A | C | −0.0438 | A | C | −0.08377116 |
| rs1250229 | CAD | T | C | 0.0516 | T | C | 0.04602593 |
| rs12524865 | CAD | A | C | −0.0805 | A | C | −0.07446373 |
| rs12597579 | CAD | T | C | −0.0536 | T | C | −0.07717893 |
| rs12603327 | CAD | T | C | −0.056 | C | T | 0.05179831 |
| rs12692735 | CAD | T | G | −0.0202 | T | G | −0.02882021 |
| rs12718465 | CAD | T | C | 0.0655 | T | C | 0.05683886 |
| rs12740374 | CAD | T | G | −0.1054 | T | G | −0.10911422 |
| rs12801636 | CAD | A | G | −0.0595 | A | G | −0.06495418 |
| rs12932445 | CAD | T | C | 0.0225 | C | T | −0.02876383 |
| rs12936587 | CAD | A | G | −0.0174 | A | G | −0.01553718 |
| rs12970066 | CAD | C | G | 0.03 | G | C | −0.03121437 |
| rs130071 | CAD | A | G | 0.0565 | A | G | 0.07129684 |
| rs13078807 | CAD | A | G | −0.1923 | G | A | 0.17744108 |
| rs1317507 | CAD | A | C | 0.0278 | A | C | 0.02955587 |
| rs13209747 | CAD | T | C | 0.0199 | T | C | 0.02745248 |
| rs1321309 | CAD | A | G | 0.0428 | A | G | 0.04157825 |
| rs13306194 | CAD | A | G | −0.0869 | A | G | −0.08883685 |
| rs13359291 | CAD | A | G | −0.0232 | G | A | 0.02295391 |
| rs1344653 | CAD | A | G | −0.0143 | G | A | 0.01367158 |
| rs1351525 | CAD | A | T | −0.0275 | A | T | −0.04067746 |
| rs13723 | CAD | A | G | −0.0311 | G | A | 0.03015594 |
| rs1378942 | CAD | A | C | −0.0335 | A | C | −0.04309328 |
| rs1412444 | CAD | T | C | 0.0754 | T | C | 0.06974109 |
| rs1421085 | CAD | T | C | −0.0361 | C | T | 0.03331057 |
| rs148910227 | CAD | T | C | −0.348 | T | C | −0.32111023 |
| rs1496653 | CAD | A | G | −0.0232 | G | A | 0.00584055 |
| rs151193009 | CAD | T | C | −0.394 | T | C | −0.44000785 |
| rs1514175 | CAD | A | G | 0.0211 | G | A | −0.01946961 |
| rs1535500 | CAD | T | G | 0.0089 | T | G | 0.01987414 |
| rs1552224 | CAD | A | C | 0.0269 | C | A | −0.04573556 |
| rs1555543 | CAD | A | C | −0.0143 | A | C | −0.01319505 |
| rs1563788 | CAD | T | C | −0.0125 | T | C | −0.01153413 |
| rs1591805 | CAD | A | G | 0.0308 | A | G | 0.02216515 |
| rs16849225 | CAD | T | C | −0.0686 | T | C | −0.06329932 |
| rs16858082 | CAD | T | C | 0.0222 | T | C | 0.04137228 |
| rs16986953 | CAD | A | G | 0.0767 | A | G | 0.07077343 |
| rs16990971 | CAD | A | G | 0.0466 | G | A | −0.04329185 |
| rs16999793 | CAD | C | G | −0.0707 | C | G | −0.06873577 |
| rs17030613 | CAD | A | C | −0.0321 | C | A | 0.02743377 |
| rs17035646 | CAD | A | G | 0.0117 | G | A | −0.01128084 |
| rs17080102 | CAD | C | G | −0.0958 | C | G | −0.11217339 |
| rs17087335 | CAD | T | G | 0.0549 | T | G | 0.05065791 |
| rs17135399 | CAD | A | G | −0.0325 | G | A | 0.03553844 |
| rs17249754 | CAD | A | G | 0.0781 | A | G | 0.0503201 |
| rs173396 | CAD | A | G | 0.0237 | A | G | 0.02244557 |
| rs17358402 | CAD | T | C | 0.0561 | T | C | 0.06422778 |
| rs17381664 | CAD | T | C | 0.1705 | C | T | −0.16932187 |
| rs174547 | CAD | T | C | −0.0147 | C | T | 0.00861122 |
| rs17465637 | CAD | A | C | −0.0915 | A | C | −0.08769559 |
| rs17477177 | CAD | T | C | −0.0386 | C | T | 0.04043499 |
| rs17514846 | CAD | A | C | 0.0799 | A | C | 0.09420911 |
| rs17612742 | CAD | T | C | −0.1001 | C | T | 0.09236533 |
| rs17678683 | CAD | T | G | −0.0716 | G | T | 0.06660256 |
| rs17695224 | CAD | A | G | 0.0108 | A | G | 0.01160008 |
| rs1800588 | CAD | T | C | 0.0211 | T | C | 0.02557299 |
| rs181360 | CAD | T | G | 0.0311 | G | T | −0.02869692 |
| rs1861411 | CAD | A | G | 0.0115 | A | G | 0.03904318 |
| rs1868673 | CAD | A | C | 0.0068 | A | C | 0.00627457 |
| rs1870634 | CAD | T | G | −0.0485 | T | G | −0.044928 |
| rs1887320 | CAD | A | G | 0.0276 | G | A | −0.03813747 |
| rs1892094 | CAD | T | C | −0.0432 | T | C | −0.04198809 |
| rs191835914 | CAD | A | C | 0.0824 | C | A | −0.0996175 |
| rs1976041 | CAD | A | G | −0.0653 | A | G | −0.06058035 |
| rs2000999 | CAD | A | G | 0.055 | A | G | 0.05721092 |
| rs200990725 | CAD | T | C | 1.095 | T | C | 1.10994771 |
| rs2021783 | CAD | T | C | −0.0484 | T | C | −0.04466016 |
| rs2057291 | CAD | A | G | 0.0378 | A | G | 0.04964609 |
| rs2066714 | CAD | T | C | −0.0475 | T | C | −0.04395988 |
| rs2068888 | CAD | A | G | −0.0177 | G | A | 0.02724399 |
| rs2075260 | CAD | A | G | 0.0253 | G | A | −0.02312772 |
| rs2075291 | CAD | A | C | 0.1283 | A | C | 0.12411373 |
| rs2107595 | CAD | A | G | 0.0598 | A | G | 0.06000351 |
| rs2128739 | CAD | A | C | 0.0918 | C | A | −0.08651522 |
| rs2144300 | CAD | T | C | −0.0263 | T | C | −0.01166838 |
| rs2145598 | CAD | A | G | −0.0193 | A | G | −0.01964328 |
| rs2156552 | CAD | A | T | 0.0242 | A | T | 0.0179407 |
| rs216172 | CAD | C | G | 0.0365 | C | G | 0.03608656 |
| rs2200733 | CAD | T | C | −0.012 | C | T | 0.01267714 |
| rs2213732 | CAD | A | G | −0.02 | G | A | 0.02027618 |
| rs2229383 | CAD | T | G | 0.0354 | G | T | −0.03518423 |
| rs2230808 | CAD | T | C | 0.0085 | T | C | 0.00284493 |
| rs2237896 | CAD | A | G | −0.0345 | A | G | −0.02645381 |
| rs2240736 | CAD | T | C | 0.0121 | T | C | 0.02049884 |
| rs2268617 | CAD | A | G | 0.0441 | G | A | −0.04398502 |
| rs2297991 | CAD | T | C | 0.0225 | T | C | 0.02380161 |
| rs2303790 | CAD | A | G | 0.0659 | G | A | −0.04853118 |
| rs2328223 | CAD | A | C | −0.0119 | C | A | 0.01080493 |
| rs2383208 | CAD | A | G | −0.0308 | G | A | 0.02686362 |
| rs2531995 | CAD | T | C | 0.0099 | T | C | 0.02860589 |
| rs2535633 | CAD | C | G | −0.0241 | G | C | 0.04603891 |
| rs2571445 | CAD | A | G | 0.03 | A | G | 0.02583162 |
| rs2575876 | CAD | A | G | −0.0417 | A | G | −0.04698913 |
| rs261967 | CAD | A | C | −0.0145 | C | A | 0.04111377 |
| rs2782980 | CAD | T | C | −0.0328 | T | C | −0.02848303 |
| rs2815752 | CAD | A | G | 0.0176 | G | A | −0.01426236 |
| rs2819348 | CAD | T | C | −0.07 | C | T | 0.06459114 |
| rs2820443 | CAD | T | C | −0.0139 | C | T | 0.00958617 |
| rs2925979 | CAD | T | C | 0.0201 | T | C | 0.02376326 |
| rs2954029 | CAD | A | T | 0.0183 | A | T | 0.02244999 |
| rs29941 | CAD | A | G | −0.0181 | G | A | 0.02200999 |
| rs3120140 | CAD | A | G | 0.0473 | A | G | 0.0269299 |
| rs3129853 | CAD | A | G | 0.0676 | A | G | 0.06921803 |
| rs3130501 | CAD | A | G | 0.0337 | A | G | 0.02554639 |
| rs326214 | CAD | A | G | −0.0088 | A | G | 0.00480729 |
| rs351855 | CAD | A | G | −0.0122 | A | G | −0.00421826 |
| rs35332062 | CAD | A | G | 0.0234 | A | G | 0.02933029 |
| rs35337492 | CAD | A | G | 0.0167 | A | G | 0.01571893 |
| rs35444 | CAD | A | G | 0.0237 | G | A | −0.04774811 |
| rs36096196 | CAD | T | C | 0.0398 | T | C | 0.03917078 |
| rs3775058 | CAD | A | T | 0.0153 | T | A | −0.01665113 |
| rs3785100 | CAD | T | C | −0.0314 | C | T | 0.03906228 |
| rs3809128 | CAD | T | C | 0.0178 | T | C | 0.01382619 |
| rs3827066 | CAD | T | C | 0.0739 | T | C | 0.06643316 |
| rs3846663 | CAD | T | C | 0.0296 | C | T | −0.03432348 |
| rs3887137 | CAD | T | C | 0.018 | T | C | 0.01863812 |
| rs4129767 | CAD | A | G | 0.019 | A | G | 0.01753188 |
| rs4148008 | CAD | C | G | −0.021 | G | C | 0.02047505 |
| rs4266144 | CAD | C | G | −0.043 | C | G | −0.03984462 |
| rs4302748 | CAD | A | G | 0.0271 | A | G | 0.0281062 |
| rs4377290 | CAD | T | C | 0.0367 | C | T | −0.04306476 |
| rs4409766 | CAD | T | C | 0.0794 | C | T | −0.07325249 |
| rs4410190 | CAD | T | C | 0.0538 | C | T | −0.05307786 |
| rs4420638 | CAD | A | G | −0.075 | G | A | 0.08531989 |
| rs4468572 | CAD | T | C | −0.0875 | T | C | −0.08073892 |
| rs459193 | CAD | A | G | −0.0175 | A | G | −0.02527039 |
| rs4593108 | CAD | C | G | 0.0598 | G | C | −0.05517929 |
| rs4613862 | CAD | A | C | 0.0315 | C | A | −0.02716256 |
| rs46522 | CAD | T | C | 0.0267 | C | T | −0.03268441 |
| rs4713766 | CAD | A | C | −0.0166 | A | C | −0.00239016 |
| rs4719841 | CAD | A | G | 0.0327 | A | G | 0.02297177 |
| rs4731420 | CAD | C | G | −0.0242 | C | G | −0.00666296 |
| rs4735692 | CAD | A | G | 0.0211 | A | G | 0.03292408 |
| rs4752700 | CAD | A | G | −0.0261 | G | A | 0.02810372 |
| rs4766228 | CAD | A | G | 0.0122 | G | A | −0.01125731 |
| rs4776970 | CAD | A | T | 0.019 | A | T | 0.03196655 |
| rs4788102 | CAD | A | G | 0.0216 | A | G | 0.04221752 |
| rs4812829 | CAD | A | G | 0.0181 | A | G | 0.0229618 |
| rs4821382 | CAD | C | G | 0.0104 | G | C | −0.00204682 |
| rs4836831 | CAD | T | C | −0.0136 | C | T | 0.01254914 |
| rs4845625 | CAD | T | C | 0.0445 | T | C | 0.04122035 |
| rs4883263 | CAD | T | C | −0.0203 | T | C | −0.01873143 |
| rs4911495 | CAD | A | C | 0.018 | C | A | −0.01344743 |
| rs4917014 | CAD | T | G | 0.0332 | G | T | −0.02619904 |
| rs4918072 | CAD | A | G | 0.0191 | A | G | 0.01762415 |
| rs499974 | CAD | A | C | 0.021 | A | C | 0.021517 |
| rs515135 | CAD | T | C | 0.0336 | T | C | 0.03100375 |
| rs5215 | CAD | T | C | −0.0201 | C | T | 0.02585252 |
| rs556621 | CAD | T | G | 0.0179 | G | T | −0.01677604 |
| rs56062135 | CAD | T | C | −0.0993 | T | C | −0.12374921 |
| rs56289821 | CAD | A | G | 0.2739 | A | G | 0.24706764 |
| rs56336142 | CAD | T | C | 0.0489 | C | T | −0.04512152 |
| rs574367 | CAD | T | G | 0.0245 | T | G | 0.06800102 |
| rs582384 | CAD | A | C | 0.0106 | A | C | 0.01005683 |
| rs590121 | CAD | T | G | 0.0486 | T | G | 0.0445939 |
| rs6038557 | CAD | A | G | 0.0284 | G | A | −0.02875574 |
| rs6065311 | CAD | T | C | −0.0458 | T | C | −0.05175701 |
| rs633185 | CAD | C | G | 0.0285 | C | G | 0.04490078 |
| rs635634 | CAD | T | C | 0.0696 | T | C | 0.06422205 |
| rs6494488 | CAD | A | G | 0.0726 | G | A | −0.06699024 |
| rs651821 | CAD | T | C | −0.0674 | C | T | 0.06219204 |
| rs663129 | CAD | A | G | 0.0494 | A | G | 0.08796346 |
| rs667920 | CAD | T | G | 0.0303 | G | T | −0.0307041 |
| rs6700559 | CAD | T | C | −0.0261 | C | T | 0.02535271 |
| rs671 | CAD | A | G | 0.1732 | A | G | 0.12328228 |
| rs6725887 | CAD | T | C | −0.0697 | C | T | 0.06431432 |
| rs6795735 | CAD | T | C | 0.0256 | C | T | −0.0236219 |
| rs6804922 | CAD | A | G | 0.0584 | G | A | −0.05388746 |
| rs6807945 | CAD | T | C | −0.0529 | C | T | 0.05076683 |
| rs6808574 | CAD | T | C | −0.014 | T | C | −0.01291823 |
| rs6813195 | CAD | T | C | −0.0082 | T | C | −0.0178775 |
| rs6818397 | CAD | T | G | 0.0302 | T | G | 0.03663864 |
| rs6829822 | CAD | T | G | 0.0251 | T | G | 0.03228224 |
| rs6882076 | CAD | T | C | −0.0225 | T | C | −0.02347432 |
| rs6905288 | CAD | A | G | 0.0359 | G | A | −0.03757368 |
| rs6909752 | CAD | A | G | 0.0643 | A | G | 0.06783203 |
| rs6960043 | CAD | T | C | −0.0119 | C | T | 0.01864762 |
| rs699 | CAD | A | G | −0.0228 | A | G | −0.03337975 |
| rs6997340 | CAD | T | C | 0.015 | C | T | −0.01178519 |
| rs702485 | CAD | A | G | 0.0331 | A | G | 0.03334703 |
| rs7087591 | CAD | A | G | −0.0325 | G | A | 0.02013776 |
| rs7120712 | CAD | A | G | 0.0679 | A | G | 0.0626534 |
| rs7178572 | CAD | A | G | −0.0287 | G | A | 0.03445457 |
| rs7185272 | CAD | C | G | 0.0405 | G | C | −0.04084457 |
| rs7199941 | CAD | A | G | 0.0399 | A | G | 0.03513851 |
| rs7202877 | CAD | T | G | 0.0312 | G | T | −0.02667102 |
| rs7206541 | CAD | A | T | −0.0552 | A | T | −0.05940826 |
| rs7208487 | CAD | T | G | −0.0215 | G | T | 0.02509522 |
| rs7225581 | CAD | A | T | 0.0585 | A | T | 0.05397974 |
| rs7258445 | CAD | A | G | −0.0559 | A | G | −0.05008695 |
| rs72654473 | CAD | A | C | −0.1769 | A | C | −0.20516668 |
| rs72689147 | CAD | T | G | −0.0677 | T | G | −0.06264442 |
| rs73015714 | CAD | C | G | −0.0812 | G | C | 0.0754775 |
| rs7304841 | CAD | A | C | 0.0153 | C | A | −0.0061072 |
| rs7306523 | CAD | A | G | −0.0159 | G | A | 0.01485534 |
| rs73069940 | CAD | C | G | 0.0226 | G | C | −0.0253497 |
| rs738409 | CAD | C | G | 0.0301 | G | C | −0.02697636 |
| rs740406 | CAD | A | G | 0.0153 | G | A | −0.00858783 |
| rs7499892 | CAD | T | C | 0.0172 | T | C | 0.0185588 |
| rs7500448 | CAD | A | G | 0.039 | G | A | −0.03598649 |
| rs7503807 | CAD | A | C | 0.0134 | C | A | −0.02452361 |
| rs751984 | CAD | T | C | 0.0348 | C | T | −0.0454369 |
| rs7525649 | CAD | T | C | 0.0462 | C | T | −0.04805102 |
| rs7560163 | CAD | C | G | 0.0225 | G | C | −0.020937 |
| rs7568458 | CAD | A | T | 0.0586 | A | T | 0.05378776 |
| rs7617773 | CAD | T | C | 0.0175 | C | T | −0.01776294 |
| rs7633770 | CAD | A | G | 0.0138 | A | G | 0.01273368 |
| rs7678555 | CAD | A | C | −0.0304 | C | A | 0.03028893 |
| rs76954792 | CAD | T | C | 0.038 | T | C | 0.05039227 |
| rs7696431 | CAD | T | G | 0.0248 | T | G | 0.02265799 |
| rs7770628 | CAD | T | C | −0.1112 | C | T | 0.11277551 |
| rs780094 | CAD | T | C | 0.0247 | C | T | −0.02300881 |
| rs7810507 | CAD | A | G | 0.0258 | A | G | 0.03150357 |
| rs7901016 | CAD | T | C | 0.0702 | C | T | −0.07072387 |
| rs7903146 | CAD | T | C | 0.039 | T | C | 0.02696928 |
| rs7916879 | CAD | A | G | 0.0242 | G | A | −0.02233008 |
| rs7955901 | CAD | T | C | −0.0138 | T | C | −0.01089063 |
| rs7980458 | CAD | T | G | −0.0538 | G | T | 0.05756393 |
| rs7989336 | CAD | A | G | 0.0085 | A | G | 0.01040071 |
| rs80234489 | CAD | A | C | 0.0135 | C | A | −0.01925215 |
| rs8030379 | CAD | A | G | 0.0174 | G | A | −0.0182544 |
| rs8042271 | CAD | A | G | −0.0782 | A | G | −0.07215753 |
| rs806215 | CAD | T | C | −0.024 | T | C | −0.03343542 |
| rs8090011 | CAD | C | G | 0.0632 | C | G | 0.04608971 |
| rs8108269 | CAD | T | G | −0.0099 | G | T | 0.00283783 |
| rs820429 | CAD | T | G | −0.0103 | G | T | 0.0245394 |
| rs838880 | CAD | T | C | 0.0135 | T | C | 0.0093594 |
| rs867186 | CAD | A | G | 0.0657 | G | A | −0.05543194 |
| rs871606 | CAD | T | C | 0.0173 | C | T | −0.01656138 |
| rs884366 | CAD | A | G | 0.0113 | A | G | 0.01055226 |
| rs885150 | CAD | T | C | −0.0433 | C | T | 0.04023012 |
| rs896854 | CAD | T | C | 0.0438 | T | C | 0.0453078 |
| rs897057 | CAD | T | C | −0.0339 | T | C | −0.03448132 |
| rs9266359 | CAD | T | C | −0.0495 | T | C | −0.04981272 |
| rs9268402 | CAD | A | G | −0.0354 | A | G | −0.03266466 |
| rs9299 | CAD | T | C | 0.0266 | C | T | −0.02853907 |
| rs9319428 | CAD | A | G | 0.0553 | A | G | 0.05678137 |
| rs9349379 | CAD | A | G | −0.189 | A | G | −0.16689235 |
| rs9357121 | CAD | T | G | 0.0867 | G | T | −0.0846812 |
| rs9367716 | CAD | T | G | −0.0212 | G | T | 0.02767625 |
| rs9376090 | CAD | T | C | 0.0133 | C | T | −0.02041753 |
| rs9390698 | CAD | A | G | 0.0265 | A | G | 0.02394689 |
| rs944172 | CAD | T | C | −0.0362 | C | T | 0.03340285 |
| rs9470794 | CAD | T | C | −0.0151 | C | T | 0.02323413 |
| rs9473924 | CAD | T | G | 0.0194 | T | G | 0.02152544 |
| rs9505118 | CAD | A | G | −0.0203 | G | A | 0.01873143 |
| rs9534262 | CAD | T | C | 0.0329 | T | C | 0.03409065 |
| rs9552911 | CAD | A | G | −0.0201 | A | G | −0.01873081 |
| rs9568867 | CAD | A | G | 0.0272 | A | G | 0.05608919 |
| rs9593 | CAD | A | T | −0.0254 | T | A | 0.02343736 |
| rs9663362 | CAD | C | G | 0.0099 | G | C | −0.00901799 |
| rs9687065 | CAD | A | G | 0.0102 | G | A | −0.02601408 |
| rs975722 | CAD | A | G | −0.0227 | G | A | 0.02989761 |
| rs9810888 | CAD | T | G | −0.0132 | G | T | 0.02698811 |
| rs9815354 | CAD | A | G | −0.0169 | A | G | −0.0101068 |
| rs9818870 | CAD | T | C | 0.0251 | T | C | 0.03506169 |
| rs9828933 | CAD | T | C | 0.0097 | C | T | −0.01862981 |
| rs9892152 | CAD | T | C | −0.0556 | T | C | −0.05147102 |
| rs9970807 | CAD | T | C | −0.106 | T | C | −0.09719914 |
| rs10051787 | BP | C | T | −0.007102 | T | C | 0.00886419 |
| rs11651052 | BP | G | A | 0.0121585 | A | G | 0.0057188 |
| rs12037987 | BP | C | T | 0.02792 | C | T | 0.02185708 |
| rs1275988 | BP | T | C | −0.037595 | T | C | −0.02721731 |
| rs12999907 | BP | G | A | −0.018735 | G | A | −0.01309656 |
| rs13041126 | BP | C | T | −0.0105835 | C | T | −0.00968047 |
| rs13143871 | BP | C | T | −0.012505 | C | T | −0.00667514 |
| rs1558902 | BP | A | T | 0.0090195 | A | T | 0.07272382 |
| rs16896398 | BP | T | A | 0.025885 | T | A | 0.02040297 |
| rs174546 | BP | T | C | 0.01284 | T | C | 0.00541147 |
| rs17843768 | BP | A | C | 0.02183 | A | C | 0.01685422 |
| rs1799945 | BP | G | C | 0.037555 | G | C | 0.03359386 |
| rs391300 | BP | C | T | −0.010165 | T | C | 0.00784806 |
| rs4336994 | BP | A | G | −0.016695 | G | A | 0.01304014 |
| rs4722766 | BP | G | C | −0.0055626 | G | C | 0.00027605 |
| rs507666 | BP | A | G | −0.0067965 | A | G | 0.0017486 |
| rs6825911 | BP | T | C | −0.01505 | C | T | 0.01184534 |
| rs7213603 | BP | C | T | 0.013248 | C | T | 0.01224508 |
| rs7405452 | BP | C | T | 0.02181 | T | C | −0.01713139 |
| rs880315 | BP | C | T | 0.031465 | T | C | −0.01852346 |
| rs93138 | BP | G | T | 0.01977 | G | T | 0.01709834 |
| rs11257655 | BMI | T | C | −0.02142 | C | T | −0.00036472 |
| rs11604680 | BMI | G | A | 0.02275 | G | A | 0.01426581 |
| rs1470579 | BMI | C | A | −0.03244 | C | A | 0.00020918 |
| rs1982963 | BMI | A | G | −0.02542 | G | A | 0.01235226 |
| rs6545814 | BMI | A | G | −0.0418 | G | A | 0.02688053 |
| rs888789 | BMI | G | A | −0.02311 | A | G | 0.01421979 |
| rs10010670 | DM | A | G | −0.0137 | G | A | 0.00178253 |
| rs10160804 | DM | A | C | 0.0278 | A | C | 0.00361711 |
| rs1029420 | DM | T | C | 0.0135 | C | T | −0.00116293 |
| rs1037814 | DM | T | C | 0.0191 | T | C | 0.00263562 |
| rs1052053 | DM | A | G | 0.0344 | G | A | −0.00447585 |
| rs10830963 | DM | C | G | −0.0134 | G | C | 0.0017435 |
| rs10886471 | DM | T | C | −0.0111 | T | C | −0.00172849 |
| rs10923931 | DM | T | G | 0.0352 | T | G | 0.005324 |
| rs11067763 | DM | A | G | −0.0115 | G | A | 0.00179726 |
| rs11624704 | DM | A | C | −0.0201 | C | A | 0.00293294 |
| rs11660468 | DM | T | C | −0.0106 | T | C | 0.00386998 |
| rs117601636 | DM | A | G | 0.1697 | G | A | −0.02257323 |
| rs1211166 | DM | A | G | 0.0204 | G | A | −0.00265428 |
| rs12229654 | DM | T | G | 0.0524 | G | T | −0.00681786 |
| rs12242953 | DM | A | G | 0.0267 | A | G | 0.003892 |
| rs12549902 | DM | A | G | 0.0731 | A | G | 0.00938577 |
| rs12571751 | DM | A | G | 0.0605 | G | A | −0.00812257 |
| rs1260326 | DM | T | C | −0.0679 | C | T | 0.00365159 |
| rs12679556 | DM | T | G | −0.0177 | T | G | −0.00230298 |
| rs12946454 | DM | A | T | −0.0281 | T | A | 0.00365614 |
| rs13233731 | DM | A | G | −0.037 | A | G | −0.00514855 |
| rs13266634 | DM | T | C | −0.1009 | T | C | −0.01338745 |
| rs13342232 | DM | A | G | −0.1273 | G | A | 0.01689765 |
| rs1334576 | DM | A | G | 0.0163 | G | A | −0.00240507 |
| rs1359790 | DM | A | G | −0.081 | A | G | −0.01034677 |
| rs1436953 | DM | T | C | −0.0716 | T | C | −0.00944977 |
| rs1532085 | DM | A | G | −0.012 | A | G | 0.00460239 |
| rs1575972 | DM | A | T | −0.1314 | A | T | −0.01709669 |
| rs16927668 | DM | T | C | 0.0111 | C | T | −0.00144424 |
| rs16967013 | DM | C | G | −0.0141 | G | C | 0.00199342 |
| rs17301514 | DM | A | G | 0.0191 | A | G | 0.00271086 |
| rs17517928 | DM | T | C | 0.0889 | T | C | 0.01156694 |
| rs17609940 | DM | C | G | −0.044 | C | G | −0.00478857 |
| rs17791513 | DM | A | G | 0.0795 | G | A | −0.00987572 |
| rs17843797 | DM | T | G | 0.0192 | G | T | −0.00249815 |
| rs1801282 | DM | C | G | 0.1424 | G | C | −0.01805139 |
| rs1832007 | DM | A | G | −0.0181 | G | A | −0.00089714 |
| rs2028299 | DM | A | C | −0.0554 | C | A | 0.00744228 |
| rs2074158 | DM | T | C | −0.0335 | C | T | 0.00435875 |
| rs2075423 | DM | T | G | −0.0391 | T | G | −0.00528802 |
| rs2081687 | DM | T | C | −0.0116 | T | C | 0.00458091 |
| rs2123536 | DM | T | C | 0.03 | T | C | 0.0041458 |
| rs2245019 | DM | A | C | −0.0253 | C | A | 0.00312462 |
| rs2258287 | DM | A | C | −0.0457 | C | A | 0.00594611 |
| rs2261181 | DM | T | C | 0.0443 | T | C | 0.00576395 |
| rs2296172 | DM | A | G | −0.0538 | G | A | 0.00700002 |
| rs2334499 | DM | T | C | 0.0138 | C | T | −0.00227208 |
| rs243019 | DM | T | C | −0.0489 | T | C | −0.00636247 |
| rs2487928 | DM | A | G | 0.014 | A | G | 0.00209745 |
| rs2642442 | DM | T | C | −0.0408 | C | T | −0.00257976 |
| rs273909 | DM | A | G | 0.0312 | G | A | −0.00250448 |
| rs2783963 | DM | A | G | −0.0198 | A | G | −0.0028939 |
| rs2796441 | DM | A | G | −0.0794 | G | A | 0.01033088 |
| rs2820315 | DM | T | C | 0.0133 | T | C | 0.00173049 |
| rs2861568 | DM | A | T | −0.0235 | A | T | −0.00505997 |
| rs2972146 | DM | T | G | 0.0563 | G | T | −0.0073253 |
| rs3213545 | DM | A | G | −0.0329 | A | G | 0.0007981 |
| rs340874 | DM | T | C | −0.0346 | C | T | 0.00494496 |
| rs35879803 | DM | A | C | 0.0483 | A | C | 0.0062844 |
| rs368123 | DM | A | G | 0.0227 | G | A | −0.00295354 |
| rs3774472 | DM | A | G | 0.0099 | G | A | −0.00128811 |
| rs3791679 | DM | A | G | 0.0169 | A | G | 0.00237446 |
| rs3810291 | DM | A | G | 0.0218 | A | G | 0.00309561 |
| rs3861086 | DM | T | C | 0.0099 | T | C | 0.00112926 |
| rs3918226 | DM | T | C | 2 | T | C | 0.26022365 |
| rs3936511 | DM | A | G | −0.0382 | G | A | 0.00497027 |
| rs4142995 | DM | T | G | 0.0076 | G | T | 0.00117992 |
| rs42039 | DM | T | C | −0.028 | T | C | −0.00321676 |
| rs4275659 | DM | T | C | −0.0405 | T | C | −0.00220803 |
| rs4458523 | DM | T | G | −0.0618 | T | G | −0.0075393 |
| rs4757391 | DM | T | C | 0.0182 | C | T | −0.00236804 |
| rs4765773 | DM | T | C | −0.0365 | T | C | −0.00518381 |
| rs4846049 | DM | T | G | −0.0111 | T | G | −0.00196258 |
| rs4923678 | DM | A | G | 0.0241 | G | A | −0.00313569 |
| rs55783344 | DM | T | C | 0.0552 | T | C | 0.00742462 |
| rs579459 | DM | T | C | −0.0295 | C | T | 0.0038383 |
| rs58542926 | DM | T | C | 0.0508 | T | C | 0.00660968 |
| rs6093446 | DM | A | G | 0.0301 | A | G | 0.0036572 |
| rs634501 | DM | A | G | 0.0388 | A | G | 0.00546635 |
| rs67156297 | DM | A | G | 0.0744 | A | G | 0.00968032 |
| rs67839313 | DM | T | C | −0.0749 | C | T | 0.00974538 |
| rs6825454 | DM | T | C | −0.0097 | C | T | 0.00157141 |
| rs6831256 | DM | A | G | −0.024 | G | A | 0.00354906 |
| rs6871667 | DM | A | G | −0.0453 | G | A | 0.00589407 |
| rs6878122 | DM | A | G | −0.0501 | G | A | 0.00492179 |
| rs6909574 | DM | A | G | −0.0179 | G | A | 0.002329 |
| rs6984210 | DM | C | G | −0.049 | G | C | 0.00637548 |
| rs702634 | DM | A | G | 0.0556 | G | A | −0.00747666 |
| rs7107784 | DM | A | G | −0.1037 | G | A | 0.01328359 |
| rs7116641 | DM | T | G | −0.0263 | G | T | 0.00378143 |
| rs7258189 | DM | T | C | −0.0169 | C | T | 0.00178088 |
| rs7403531 | DM | T | C | 0.058 | T | C | 0.00787254 |
| rs748431 | DM | T | G | −0.0211 | T | G | −0.00274536 |
| rs7528419 | DM | A | G | −0.0203 | G | A | 0.00264127 |
| rs7610618 | DM | T | C | 0.0486 | T | C | 0.00632343 |
| rs7616006 | DM | A | G | −0.0148 | G | A | −0.00048199 |
| rs769449 | DM | A | G | −0.0364 | A | G | −0.00473607 |
| rs78169666 | DM | A | C | 0.1398 | C | A | −0.01818963 |
| rs7897379 | DM | T | C | −0.0116 | C | T | 0.00616915 |
| rs7917772 | DM | A | G | 0.0109 | A | G | 0.00153526 |
| rs79223353 | DM | A | G | −0.0416 | A | G | −0.00556314 |
| rs79548680 | DM | C | G | 0.0547 | C | G | 0.00694155 |
| rs820430 | DM | A | G | −0.0095 | A | G | −0.00123606 |
| rs840616 | DM | T | C | −0.0374 | T | C | −0.00454849 |
| rs9309245 | DM | C | G | −0.0313 | G | C | 0.0040725 |
| rs9512699 | DM | A | G | −0.033 | G | A | 0.00429369 |
| rs9591012 | DM | A | G | 0.0148 | A | G | 0.00144076 |
| rs984222 | DM | C | G | 0.0098 | C | G | 0.0012751 |
| rs10401969 | TC | C | T | −0.05997 | C | T | −0.00853347 |
| rs10889353 | TC | C | A | −0.05622 | C | A | −0.00826634 |
| rs11136341 | TC | A | G | −0.0418 | G | A | 0.00614609 |
| rs117711462 | TC | A | G | 0.2115 | A | G | 0.03109802 |
| rs12027135 | TC | A | T | −0.0292 | T | A | 0.00456097 |
| rs12453914 | TC | A | C | 0.01427 | A | C | 0.0020982 |
| rs12927205 | TC | A | G | 0.0666 | G | A | −0.00979257 |
| rs13115759 | TC | A | T | −0.011 | A | T | −0.00161739 |
| rs1367117 | TC | A | G | 0.05277 | A | G | 0.00775907 |
| rs1495741 | TC | A | G | −0.01817 | A | G | −0.00300605 |
| rs16844401 | TC | A | G | 0.02292 | A | G | 0.00396363 |
| rs17122278 | TC | A | G | −0.0469 | G | A | 0.00689597 |
| rs181359 | TC | A | G | −0.01433 | A | G | −0.00210702 |
| rs2000813 | TC | T | C | 0.02996 | T | C | 0.00440518 |
| rs2244608 | TC | G | A | 0.02163 | G | A | 0.00318038 |
| rs2302593 | TC | G | C | −0.01066 | G | C | −0.00189345 |
| rs247616 | TC | T | C | 0.05349 | T | C | 0.00766428 |
| rs4883201 | TC | G | A | −0.0245 | G | A | −0.00360237 |
| rs5996074 | TC | A | G | −0.01452 | G | A | 0.00226036 |
| rs7134594 | TC | T | C | 0.01965 | T | C | 0.00288925 |
| rs7258950 | TC | G | A | 0.05438 | A | G | −0.00799579 |
| rs737337 | TC | C | T | −0.04973 | C | T | −0.00779697 |
| rs7965082 | TC | T | C | −0.02394 | T | C | −0.00352003 |
| rs964184 | TC | C | G | −0.05343 | G | C | 0.00785611 |
| rs10203174 | Stroke | C | T | −0.066 | T | C | 0.00055178 |
| rs1050362 | Stroke | C | A | −0.02 | A | C | 0.00016721 |
| rs10947231 | Stroke | C | A | 0.032 | A | C | −0.00026753 |
| rs11634397 | Stroke | A | G | −0.025 | G | A | 0.00020901 |
| rs11957829 | Stroke | A | G | 0.163 | G | A | −0.00136272 |
| rs12500824 | Stroke | G | A | 0.017 | A | G | −0.00014212 |
| rs12607689 | Stroke | G | T | 0.024 | T | G | −0.00020065 |
| rs3702 | Stroke | T | C | 0.042 | C | T | −0.00035113 |
| rs1424233 | Stroke | T | C | −0.015 | C | T | 0.0001254 |
| rs1467605 | Stroke | C | A | 0.029 | A | C | −0.00024245 |
| rs1508798 | Stroke | T | C | 0.033 | C | T | −0.00027589 |
| rs16933812 | Stroke | T | G | 0.016 | G | T | −0.00013376 |
| rs17080091 | Stroke | C | T | 0.146 | T | C | −0.0012206 |
| rs17608766 | Stroke | T | C | −0.091 | C | T | 0.00076078 |
| rs180327 | Stroke | T | C | 0.026 | C | T | −0.00021737 |
| rs1878406 | Stroke | C | T | −0.061 | T | C | 0.00050998 |
| rs2075650 | Stroke | A | G | 0.038 | G | A | −0.00031769 |
| rs2107732 | Stroke | G | A | 0.147 | A | G | −0.00122896 |
| rs2237892 | Stroke | C | T | 0.054 | T | C | −0.00045145 |
| rs2295786 | Stroke | A | T | 0.062 | A | T | 0.00051834 |
| rs246600 | Stroke | C | T | 0.082 | T | C | −0.00068554 |
| rs2625967 | Stroke | G | A | −0.013 | G | A | −0.00010868 |
| rs2758607 | Stroke | G | A | 0.044 | A | G | −0.00036785 |
| rs2972143 | Stroke | G | A | 0.027 | A | G | −0.00022573 |
| rs34008534 | Stroke | A | G | 0.053 | G | A | −0.00044309 |
| rs35419456 | Stroke | C | A | −0.265 | A | C | 0.00221547 |
| rs376563 | Stroke | C | T | −0.027 | T | C | 0.00022573 |
| rs4471613 | Stroke | G | A | 0.042 | A | G | −0.00035113 |
| rs4724806 | Stroke | C | G | 0.026 | G | C | −0.00021737 |
| rs4777561 | Stroke | C | T | 0.03 | T | C | −0.00025081 |
| rs4939883 | Stroke | C | T | −0.037 | T | C | 0.00030933 |
| rs60154123 | Stroke | C | T | −0.032 | T | C | 0.00026753 |
| rs6544713 | Stroke | C | T | 0.108 | T | C | −0.00090291 |
| rs7136259 | Stroke | C | T | 0.064 | T | C | −0.00053506 |
| rs7193343 | Stroke | C | T | −0.043 | C | T | −0.00035949 |
| rs73596816 | Stroke | G | A | −0.176 | A | G | 0.00147141 |
| rs736699 | Stroke | A | G | 0.125 | G | A | −0.00104503 |
| rs7859727 | Stroke | T | C | 0.068 | C | T | −0.0005685 |
| rs7947761 | Stroke | A | G | −0.05 | G | A | 0.00041801 |
| rs832552 | Stroke | G | T | −0.04 | T | G | 0.00033441 |
| rs1077834 | LDL | C | T | −0.02867 | C | T | 0 |
| rs10820405 | LDL | A | G | 0.0258 | A | G | 0 |
| rs17145738 | LDL | T | C | 0.04998 | T | C | 0 |
| rs1883025 | LDL | T | C | −0.02659 | T | C | 0 |
| rs9916693 | LDL | A | T | 0.01966 | A | T | 0 |
| rs11066280 | HDL | A | T | −0.077 | A | T | 0 |
| rs11196288 | HDL | G | A | 0.009104 | G | A | 0 |
| rs11787335 | HDL | T | C | −0.008877 | T | C | 0 |
| rs12202017 | HDL | G | A | −0.01248 | G | A | 0 |
| rs12493885 | HDL | C | G | −0.1558 | G | C | 0 |
| rs12535846 | HDL | A | G | 0.0108 | A | G | 0 |
| rs12897 | HDL | A | G | 0.0142 | A | G | 0 |
| rs13277801 | HDL | T | C | −0.0111 | C | T | 0 |
| rs1689800 | HDL | G | A | −0.02752 | G | A | 0 |
| rs17150703 | HDL | A | G | 0.008758 | A | G | 0 |
| rs1800234 | HDL | C | T | 0.0435681 | C | T | 0 |
| rs1867624 | HDL | T | C | 0.0182 | C | T | 0 |
| rs1902859 | HDL | C | T | 0.01537 | C | T | 0 |
| rs2415317 | HDL | A | G | −0.0126 | A | G | 0 |
| rs35432 | HDL | T | C | 0.006571 | T | C | 0 |
| rs4932370 | HDL | A | G | 0.0444 | A | G | 0 |
| rs6537746 | HDL | A | G | 0.009876 | G | A | 0 |
| rs660599 | HDL | A | G | −0.01626 | A | G | 0 |
| rs7228667 | HDL | T | C | 0.004878 | C | T | 0 |
| rs9854454 | HDL | T | C | 0.02358 | T | C | 0 |
| rs990620 | HDL | G | A | −0.007548 | A | G | 0 |
| rs157582 | TG | T | C | 0.04522 | T | C | 0 |
| rs2292318 | TG | T | C | −0.02176 | T | C | 0 |
| rs312949 | TG | G | C | −0.01225 | C | G | 0 |
| rs439401 | TG | C | T | 0.0687205 | C | T | 0 |

TABLE 5
Weights of subphenotypes in the comprehensive polygenic
risk score for coronary artery disease

| Subphenotype | PRS weight |
|---|---|
| Coronary artery disease | 0.452 |
| Blood pressure | 0.074 |
| Body mass index | 0.072 |
| Diabetes | 0.064 |
| Total cholesterol | 0.038 |
| Stroke | 0.004 |
| Low density lipoprotein | 0 |
| (LDL) cholesterol | |
| High density lipoprotein | 0 |
| (HDL) cholesterol | |
| Triglycerides | 0 |

### Statistical Analysis

For continuous variables, population characteristics were described as mean (standard deviation); for categorical variables, population characteristics were described as number (percentage). Polygenic genetic scores were categorized into three groups (high, medium, and low genetic risk groups) according to <20%, 20%-80%, and >80% quartiles. Cox proportional risk regression models adjusted for age and sex, corrected for cohort origin, and accounting for competing risks of non-coronary artery disease death were used to estimate hazard ratios (HRs) for coronary artery disease events and their 95% confidence intervals (CIs) for different genetic risk groups. A Cox proportional risk regression model with age as the time scale was used to evaluate the lifetime risk (up to the age of 80) of coronary artery disease in different genetic risk subgroups. A 10-year cardiovascular disease risk score was calculated for each individual using the China-PAR formula, and they were then categorized into low, medium, and high clinical risk groups with cutoffs of <5%, 5-9.9%, and ≥10%. In addition, the Cox proportional risk model was used to calculate the 10-year risk of coronary artery disease and the lifetime risk after accounting for competing risks in people in different age brackets using the Cox proportional risk model, and both the China-PAR clinical risk scores and the genetic risk scores were entered into the model as categorical variables with the aim of developing a simple and practical coronary artery disease risk evaluation chart (RISK CHART). The ‘survfit.coxph’ function from the R package survival was used in the analysis. All reported p-values in this study were not corrected, and a p-value <0.05 on both sides was considered statistically significant. Statistical analyses were performed in the R software (R Foundation for Statistical Computing, Vienna, Austria, version 3.5.0) or the SAS statistical software (SAS Institute Inc, Cary, NC, version 9.4).

### Baseline Information for Prospective Cohort

Table 6 shows the baseline information of the 41,271 study subjects in the cohort population. The mean age at baseline was 52.3 years (with a standard deviation of 10.6 years), of which 42.5% were male. Men had a higher prevalence of current smoking compared to women. After a total of 534,701 years of follow-up (with an average of 13.0 years of follow-up), 1,303 cases of coronary artery disease occurred.

TABLE 6
Baseline information for prospective cohort

| | Total | | Male | | Female | |
|---|---|---|---|---|---|---|
| | (N = 41,271) | | (N = 17,560) | | (N = 23,771) | |
| Baseline age, years | 52.3 | (10.6) | 52.8 | (10.8) | 51.9 | (10.5) |
| Current smokers, N (%) | 10,026 cases | (24.4%) | 9,380 cases | (53.5%) | 646 cases | (2.7%) |
| Family history of coronary | 2,255 cases | (5.5%) | 965 cases | (5.5%) | 1,290 cases | (5.4%) |
| artery disease, N (%) | | | | | | |
| Body mass index, kg/m² | 23.8 | (3.6) | 23.4 | (3.4) | 24.1 | (3.8) |
| Systolic blood pressure, mmHg | 128.4 | (21.9) | 129.1 | (20.9) | 128.02 | (22.6) |
| Diastolic blood pressure, | 79.4 | (11.9) | 80.6 | (12.0) | 78.5 | (11.8) |
| mmHg | | | | | | |
| Total cholesterol, mg/dl | 180.5 | (36.3) | 177.9 | (36) | 182.4 | (36.5) |
| Blood glucose, mg/dl | 94.2 | (27.2) | 93.2 | (25.4) | 94.9 | (28.4) |
| Hypertension, N (%) | 14,038 cases | (34%) | 6,187 cases | (35.2%) | 7,851 cases | (33.1%) |
| Diabetes, N (%) | 2,705 cases | (6.8%) | 1,012 cases | (6%) | 1,693 cases | (7.4%) |
| Dyslipidemia, N (%) | 13,399 cases | (33%) | 6,063 cases | (35.2%) | 7,336 cases | (31.5%) |
| Number of new coronary | 1303 | (3.2%) | 635 | (3.6%) | 668 | (2.8%) |
| events, N (%) | | | | | | |
| Years of follow-up | 13.0 | (4.8) | 12.9 | (5.1) | 13.0 | (4.6) |

Values in mean (SD) or N (%). CAD, coronary artery disease.

### Prediction of Coronary Artery Disease by Polygenic Risk Scores

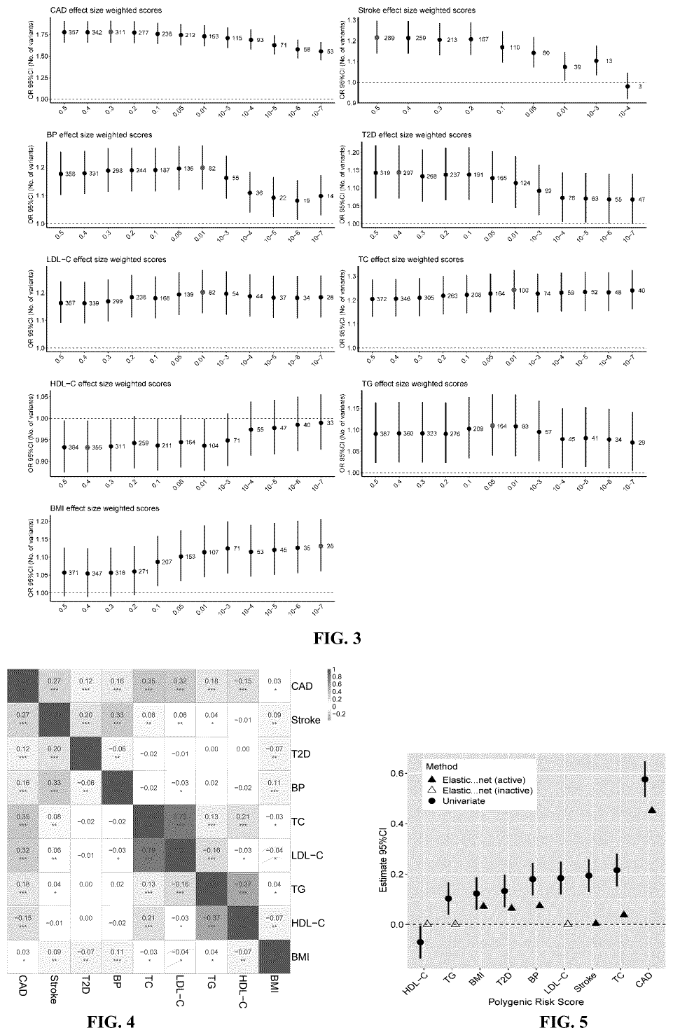

12 combinations of different SNPs were first selected in the present invention by 12 thresholds (0.5, 0.4, 0.3, 0.2, 0.1, 0.05, 0.01, 10⁻³, 10⁻⁴, 10⁻⁵, 10⁻⁶, 10⁻⁷) set based on the P-values of the coronary artery disease GWAS results of East Asian populations. Then, the PRSs for coronary artery disease were calculated by using data of the GWAS results of European populations as the SNP effect sizes in the training set, and further evaluated for the degree of association with coronary artery disease thereof. As shown in FIG. 2, compared with using GWAS effect sizes for coronary artery disease in the East Asian population, the OR (95% CI) values of the association with coronary artery disease for all 12 PRSs incorporating different combinations of SNPs (per SD increment) were significantly decreased when using effect sizes from the European population. Therefore, in this study, GWAS effect sizes from East Asian populations were used to establish respective subphenotypic PRSs, and the degree of association between each candidate subphenotypic PRS and coronary artery disease in the training set is shown in FIG. 3. The score with the largest OR value was selected as the final subphenotypic PRS.

With the best coronary artery disease subphenotypic (CAD) PRS, a set of coronary artery disease risk-related genes associated with East Asian populations was identified, including 311 CAD-associated single-nucleotide polymorphisms (SNPs) as shown in Table 4. The risk of developing coronary artery disease in an East Asian population can be well evaluated by detecting these CAD-associated SNPs and obtaining the genetic risk scores for the risk of incidence with Σβi×Ni. The effect sizes of each CAD-associated each SNP can be normalized by using the effect sizes of SNPs in the subphenotypic PRS column in Table 4, or by using the effect sizes of SNPs in the metaPRS column in Table 4. The higher the genetic risk score, the higher the individual's risk of developing coronary artery disease is.

There were different degrees of correlations between the 9 subphenotypic PRS (FIG. 4). The association between the 9 subphenotypic PRS and coronary artery disease was further evaluated using an elastic net logistic regression model which could correct the correlation between the individual subphenotypic PRS. The ORs estimated by elastic net logistic regression are shown in FIG. 5 in comparison with those estimated by univariate logistic regression (LDL-C, TG and HDL-C are weighted as 0 in FIG. 5).

With the protocol for evaluating the risk of developing coronary artery disease of the present invention, based on the detection of 311 CAD-associated SNPs shown in Table 4, by further selectively detecting one or more groups of SNPs among the 21 BP-associated SNPs, 6 BMI-associated SNPs, 108 DM-associated SNPs, 24 TC-associated SNPs, and 40 Stroke-associated SNPs shown in Table 4, a genetic risk score for the risk of incidence is obtained by Σβi×Ni, and the risk of coronary artery disease in East Asian populations could be better evaluated. When the protocol for evaluating the risk of developing coronary artery disease of the present invention includes the detection of one or more groups of BP, BMI, DM, TC, and Stroke-associated SNPs, the effect sizes of these SNPs may be uniformly used as the effect sizes of the SNPs in the subphenotypic PRS column of Table 4, and it is preferred to uniformly use the effect sizes of the SNPs in the metaPRS column of Table 4. The higher the genetic risk score, the higher the individual's risk of developing coronary artery disease.

The present invention also establishes a metaPRS for coronary artery disease by integrating the nine subphenotypic PRSs and validating in a cohort population.

The degree of the association between metaPRS and the coronary artery disease risk was the highest for the metaPRS compared with the subphenotypic PRSs (FIG. 6), with an HR for coronary artery disease of 1.44 (95% CI: 1.36-1.52) per 1 SD increment in metaPRS (P=2.84×10⁻³⁹). The association between metaPRS and coronary artery disease was independent of dyslipidemia, hypertension, BMI, diabetes, smoking status, and family history of coronary artery disease (Table 7).

TABLE 7
metaPRS after correction for coronary risk factors and hazard
ratios for coronary events (per 1 SD increment in metaPRS)

| Model | HR | (95% CI) | P value |
|---|---|---|---|
| metaPRS | 1.44 | (1.36, 1.52) | 2.84 × 10⁻³⁹ |
| metaPRS + dyslipidemia | 1.42 | (1.34, 1.50) | 2.54 × 10⁻³⁵ |
| metaPRS + hypertension | 1.41 | (1.34, 1.49) | 2.78 × 10⁻³⁵ |
| metaPRS + diabetes | 1.43 | (1.36, 1.51) | 1.33 × 10⁻³⁷ |
| metaPRS + body mass index | 1.42 | (1.35, 1.50) | 1.74 × 10⁻³⁶ |
| metaPRS + smoking | 1.44 | (1.36, 1.52) | 4.55 × 10⁻³⁹ |
| metaPRS + Family | 1.44 | (1.36, 1.52) | 9.52 × 10⁻³⁹ |
| history of CAD | | | |
| metaPRS + 6 common | 1.39 | (1.32, 1.47) | 2.75 × 10⁻³¹ |
| CAD risk factors | | | |

CAD, coronary artery disease; PRS, polygenetic risk score; HR, hazard ratio; CI, confidence interval.

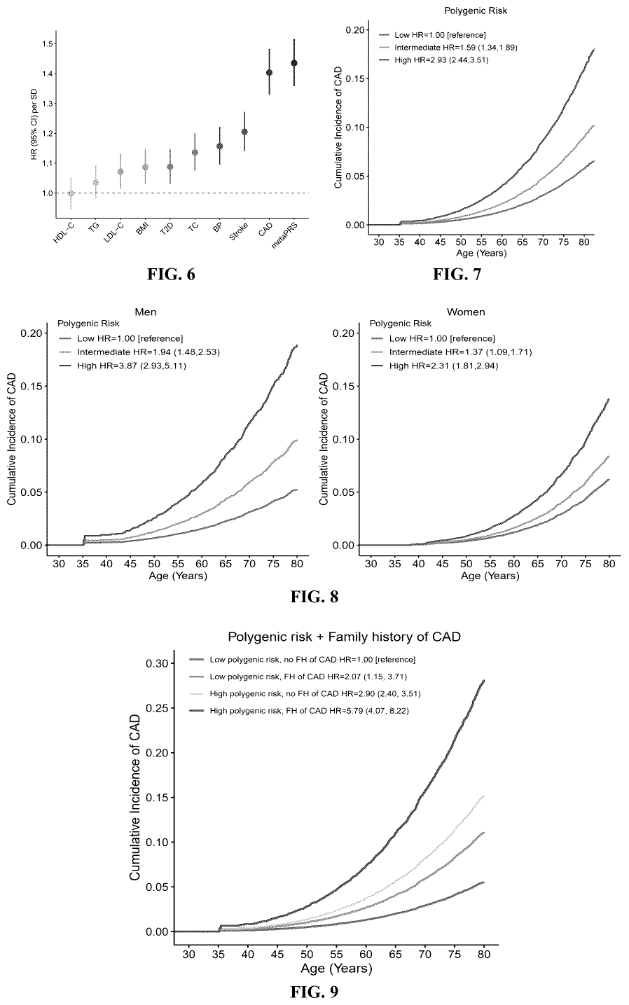

The metaPRSs are divided into groups of 20% and 80% quartiles, individuals with a high genetic risk (upper 80% in genetic risk) had a 3-fold higher risk of the occurance of a coronary artery disease event (HR=2.93, 95% CI: 2.44-3.51) compared with individuals with a low genetic risk (lower 20% in genetic risk) (FIG. 7). The cumulative risk of developing coronary artery disease by the age of 80 in these two groups was 5.8% and 16.0%, respectively. Analyses stratified by sex yielded similar results (FIG. 8). Further refined stratification of coronary artery disease risks would be facilitated if both the genetic risk and the family history of coronary artery disease were considered. For example, in a population with a low genetic risk and no family history, the lifetime risk of coronary artery disease was 5.6%; however, if high genetic risk and family history were combined, the lifetime risk of coronary artery disease became 28.2%, with a 5.79-time difference (FIG. 9).

TABLE 8
Quick checklist for genetic risk stratification

| | | Medium | Medium | Medium | |
|---|---|---|---|---|---|
| | | (20%- | (40%- | (60%- | High |
| Group | <20% | 40%) | 60%) | 80%) | (>80%) |
| Genetic | <−0.186 | −0.186~0.110 | 0.110~0.363 | 0.363~0.650 | >0.650 |
| risk | | | | | |
| score | | | | | |

### Coronary Artery Disease Risk Stratification by Combining Polygenic Genetic Risk and Clinical Risk

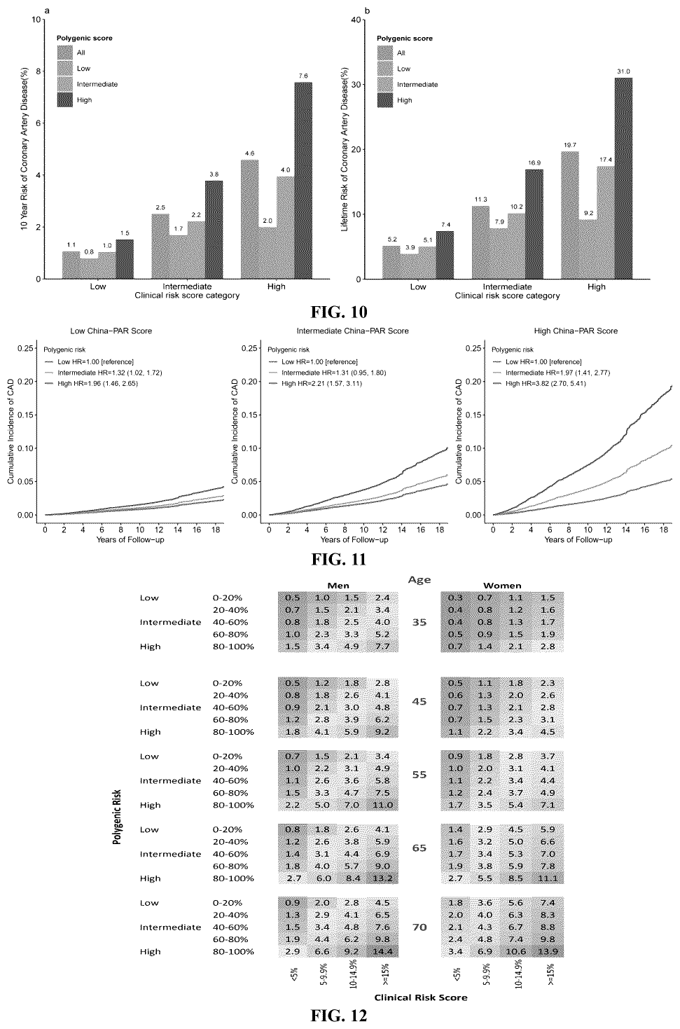

The potential for re-stratification of the risk of coronary artery disease (CAD) considering a clinical risk score (10-year cardiovascular risk score of China-PAR) in combination with the genetic risk was evaluated in the present invention. It was observed that the genetic risk played an important role in the re-stratification of both the 10-year incidence risk as well as the lifetime incidence risk of CAD in each China-PAR group (FIG. 10), and there could be a potential interaction between the genetic risk score and the China-PAR score (P=0.02). In particular, the relative risk between the high and low genetic risk groups was greater in the high China-PAR score group (HR: 3.82; 95% CI: 2.70-5.41) than in the low China-PAR score group (HR: 1.96; 95% CI: 1.46, 2.65) (FIG. 11). Similar differences were found when calculating an absolute risk, with the 10-year cumulative incidence of coronary artery disease in the high China-PAR score population being 2.0% and 7.6% for the low and high genetic risk groups, respectively; their corresponding lifetime risks of coronary artery disease were 9.2% and 31.0%, respectively. In those with high clinical risk but low genetic risk, the 10-year and lifetime risks of coronary artery disease were lower than the average risk values in those with moderate clinical risk. It is more clinically significant that individuals at a medium clinical risk and a high genetic risk had similar 10-year and lifetime risks of coronary artery disease (10-year risk of 3.8% and lifetime risk of 16.9%) as those at a high clinical risk and medium genetic risk (10-year risk of 4.0% and lifetime risk of 17.4%).

### Coronary Artery Disease Risk Evaluation Scale Based on Genetic and Clinical Risk

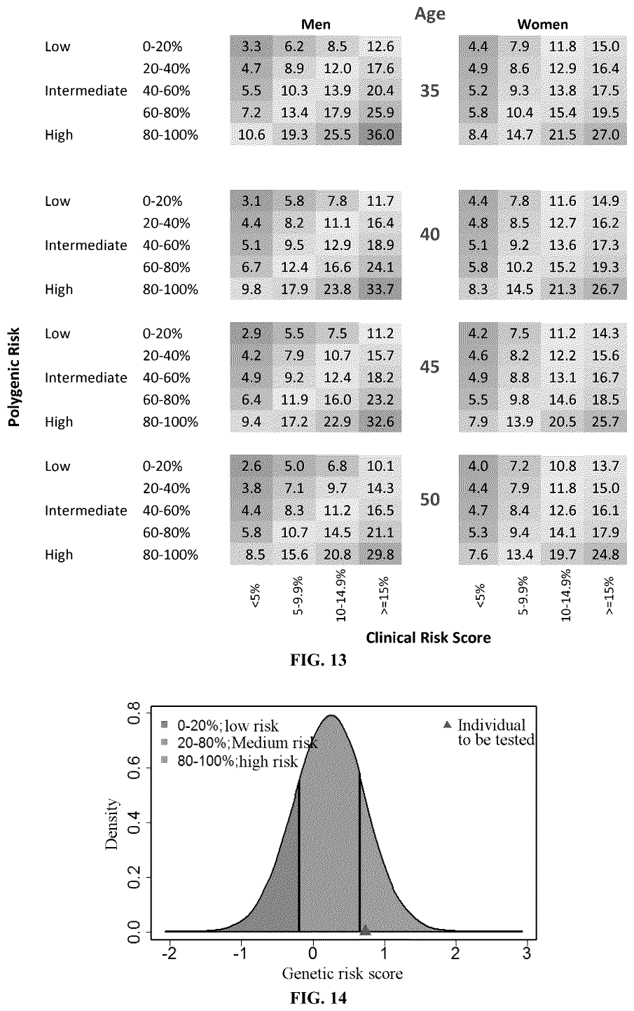

In order to increase the utility of the present invention, a simple evaluation chart that integrates both the genetic score and the clinical score has been developed in the present invention. It was found that the genetic score was able to further refine and re-stratify the absolute risk of developing coronary artery disease on the basis of the clinical score (FIGS. 12 and 13). For example, for men aged 65 to 69 with a clinical risk of coronary artery disease ≥15%, the corresponding 10-year risk of developing coronary artery disease was influenced by genetic factors with a range variation of 4.1% to 13.2%; the corresponding 10-year risk of developing coronary artery disease in women could range from 5.9% to 11.1%. Similarly, lifetime risk of coronary artery disease increased significantly with increasing genetic risk under any clinical risk stratefication, reaching 36% and 27% respectively for men or women aged 35 to 39 with a combined high genetic risk and high clinical risk. It is noteworthy that for those at a medium clinical risk and a combined high genetic risk, the 10-year or lifetime risk of coronary artery disease thereof could exceed the average level in those at a high clinical risk (clinical risk of 10-14%).

### Method for Calculating the 10-Year Risk of ASCVD by China-PAR Model

### The Calculations of the Model are Briefly Summarized Below:

the 10-year risk prediction inclusion variables for the development of ASCVD in men and women and their parameters are shown in Table 9.

TABLE 9
Variables required for the ASCVD 10-year risk
prediction model and corresponding parameters

| Variable | Male | Female |
|---|---|---|
| Ln (age), years | 31.97 | 24.87 |
| Ln (post-treatment systolic blood pressure), mmHg | 27.39 | 20.71 |
| Ln (untreated systolic blood pressure), mmHg | 26.15 | 19.98 |
| Ln (total cholesterol), mg/dL | 0.62 | 0.16 |
| Ln (high-density lipoprotein cholesterol), mg/dL | −0.69 | −0.22 |
| Ln (waist), cm | −0.71 | 1.48 |
| Smoking (1 = Yes, 0 = No) | 3.96 | 0.49 |
| Diabetes (1 = Yes, 0 = No) | 0.36 | 0.57 |
| Place of residence (1 = North, 0 = South) | 0.48 | 0.54 |
| Rural-urban (1 = urban, 0 = rural) | −0.16 | N/A |
| Family history of ASCVD (1 = yes, 0 = no) | 6.22 | N/A |
| Ln (age) × smoking | −0.94 | N/A |
| Ln (age) × Ln (post-treatment systolic blood pressure) | −6.02 | −4.53 |
| Ln (age) × Ln (untreated systolic blood pressure) | −5.73 | −4.36 |
| Ln (age) × family history of ASCVD (1 = yes, 0 = no) | −1.53 | N/A |
| MeanX'B | 140.68 | 117.26 |
| Baseline 10-year survival | 0.97 | 0.99 |

Note:
Ln, natural logarithmic transformation; N/A, the variable was not included in the model; MeanX'B, mean of the sum of the products of each variable and its parameter of the population in this study; ASCVD, atherosclerotic cardiovascular disease.

For an adult, with the known specific values of his or her age, treated or untreated systolic blood pressure level, and other variables, by multiplying the parameters corresponding to the different variables in Table 9, IndX′B (i.e., the sum of the products of the specific values of the variables and the corresponding parameters for the adult) can be calculated, and the 10-year risk for the onset of ASCVD can be obtained by substituting IndX′B into the following equation:

$$1 - {{S}_{10}}^{exp ( {IndX}^{′} B - {MeanX}^{′} B )}$$

wherein, S₁₀ is the baseline 10-year survival rate, which is 0.97 for men and 0.99 for women; MeanX′B is the “mean of the sum of the products of each variable and its parameter of the population in this study”, which is 140.68 for men and 117.26 for women (see Table 9); IndX′B is the sum of the product of the specific values of each variable and the corresponding parameter (see table above) for a given individual.

### Example 2

### Practical Application Case 1:

An individual to be tested, Li, a Chinese Han people, was evaluated for the genetic risk of developing coronary artery disease using the testing device for evaluating a genetic risk of coronary artery disease of the present invention, and then given guidance and advices. The following steps were essentially conducted: collecting fasting blood, isolating DNA from anticoagulated blood of the individual to be tested, and utilizing an Illumina Hiseq X Ten sequencer to detect the genotypes of a plurality of loci of Li, including the aforementioned 510 loci of the present invention.

The results of each SNP were compared with Table 4 to find the genetic contribution of the corresponding effect allele at each locus, weighted and summed to obtain the Genetic risk score=Σμi×Ni. The genetic risk score for coronary artery disease for Li was calculated to be 0.730, and was distributed in the population with a high genetic risk for coronary artery disease according to Table 8 (80% to 100%) (FIG. 14). The lifetime risk of coronary artery disease in this population (up to the age of 80) was 16.0%.

Li had a high genetic risk of coronary artery disease and was advised to develop and maintain strictly a good lifestyle and behavioral habits such as no smoking, controlling weight, increasing physical activities, and keeping a healthy diet; if risk factors such as hypertension, hyperlipidemia, and diabetes were present, the blood pressure, blood lipids, and blood glucose levels should be strictly controlled under the guidance of a clinician. Physical examination should be conducted at least once a year and the risk of cardiovascular and cerebrovascular diseases should be further evaluated.

### Practical Application Case 2:

The individual to be tested, Li, a Chinese Han people, male, 45 years old, had a systolic blood pressure of 160 mmHg, a total cholesterol of 280 mg/dl, a high-density lipoprotein cholesterol of 80 mg/dl, a waist circumference of 85 cm, was a smoker, suffered from diabetes mellitus, lived in a rural area in northern China, and has a combined family history of atherosclerotic cardiovascular disease. Li was evaluated for the genetic risk of developing coronary artery disease using the testing device for evaluating genetic risk of coronary artery disease of the present invention, and was given guidance and advices in combination with the China-PAR clinical risk score. The following steps were essentially conducted: collecting fasting blood, isolating DNA from anticoagulated blood of the individual to be tested, and utilizing an Illumina Hiseq X Ten sequencer to detect the genotypes of a plurality of loci of Li, including the aforementioned 510 loci of the present invention.

Genetic risk evaluation: Li's test results were analyzed and processed, and the results of each SNP were compared with Table 4 to find the genetic contribution of the corresponding effect allele at each locus, weighted and summed to obtain the Genetic risk score=Σβi×Ni. The genetic risk score for coronary artery disease for Li was calculated to be 0.730, and was distributed in the population with a high genetic risk for coronary artery disease according to Table 8 (80% to 100%) (FIG. 14).

Clinical risk evaluation: based on the China-PAR clinical risk model and calculated according to the model parameters provided in Table 9, Li's 10-year risk of ASCVD was 17.7%, which was in the high clinical risk group.

With the genetic and clinical risks combined, Li, male, 45 years old, had a high genetic risk (80%-100%) in combination with a high clinical risk (>15%). With reference to FIGS. 12 and 13, Li had a 10-year risk of coronary artery disease of 9.2% and a lifetime risk of coronary artery disease of 32.6%. Therefore, he was advised to develop and maintain strictly a good lifestyle and behavioral habits, such as no smoking, controlling weight, increasing physical activities, and keeping a healthy diet; and blood pressure, lipid, and blood glucose levels should be strictly controlled under the guidance of clinicians. Physical examination should be conducted at least once a year and coronary artery disease risk should be further evaluated.

### Practical Application Case 3:

The individual to be tested in the above Application Case 1, Li, if the individual's information was: Chinese Han people, male, 45 years old, systolic blood pressure of 145 mmHg, total cholesterol of 280 mg/dl, HDL cholesterol of 80 mg/dl, waist circumference of 85 cm, smoker, suffering from diabetes, and residing in a rural area in northern China.

Genetic risk evaluation was carried out as follows: Li's test results were analyzed and processed, and the results of each SNP were compared with Table 4 to find the genetic contribution of the corresponding effect allele at each locus, weighted and summed to obtain the Genetic risk score=Σβi×Ni. The genetic risk score for coronary artery disease for Li was calculated to be 0.730, and was distributed in the population with a high genetic risk for coronary artery disease according to Table 8 (80% to 100%) (FIG. 14).

Clinical risk evaluation was carried out as follows: based on the China-PAR clinical risk model and calculated according to the model parameters provided in Table 9, Li's 10-year risk of ASCVD was 8.3%, which was in the medium clinical risk group.

With the clinical risk and genetic risk combined, Li, male, 45 years old, had a high genetic risk (80%-100%) in combination with a medium clinical risk (5% to 9.9%). With reference to FIGS. 12 and 13, Li had a 10-year risk of coronary artery disease of 4.1% and a lifetime risk of coronary artery disease of 17.2%. Although Li had a medium clinical risk, in combination with the genetic score, his risk of coronary artery disease was similar to or even higher than that of those in the population with a high clinical risk (clinical risk within the 10%-14.9% range). Therefore, in addition to strict adherence to a healthy lifestyle, enhanced management of blood pressure, blood glucose, and blood lipids according to clinical guidelines was advised.

### Practical Application Case 4:

The individual to be tested in the aforementioned Application Case 1, Li, if the individual's information was: Chinese Han people, male, 35 years old, with a combined family history of coronary artery disease.

Genetic risk evaluation was carried out as follows: Li's test results were analyzed and processed, and the results of each SNP were compared with Table 4 to find the genetic contribution of the corresponding effect allele at each locus, weighted and summed to obtain the Genetic risk score=Σβi×Ni. The genetic risk score for coronary artery disease for Li was calculated to be 0.730, and was distributed in the population with a high genetic risk for coronary artery disease according to Table 8 (80% to 100%) (FIG. 14). The lifetime risk of coronary artery disease (up to the age of 80) in this population was 16.0%.

Li had a high genetic risk (>80%) and a combined family history of coronary artery disease, and Li's lifetime risk of coronary artery disease was 28.2% according to FIG. 9. The combination of the genetic risk and family history predicted a high risk of coronary artery disease in Li, and it was advised that in addition to healthy lifestyle management, he could pay particular attention to controlling the blood pressure, blood glucose, blood lipids, and body weight, perform health examination regularly, and seek medical help in case of any abnormality.

1. A method for evaluating a risk of developing a coronary artery disease, comprising:
detecting a sample from an individual to obtain the individual's information, wherein the individual is from an East Asian population, and wherein the individual's information comprises the following single nucleotide polymorphism locus information:
CAD-associated single nucleotide polymorphism loci: rs10064156, rs10071096, rs10093110, rs10096633, rs10139550, rs10237377, rs10260816, rs10267593, rs1027087, rs10278336, rs10455782, rs10503675, rs10512861, rs10513801, rs10745332, rs10757274, rs10773003, rs10842992, rs10846744, rs10857147, rs10890238, rs10953541, rs10968576, rs11030104, rs11057830, rs11067762, rs11077501, rs11099493, rs11107829, rs11125936, rs11142387, rs1116357, rs11170820, rs11205760, rs11206510, rs11509880, rs11556924, rs11557092, rs115696548, rs11601507, rs11677932, rs1169288, rs1173766, rs11787792, rs11810571, rs11838267, rs11838776, rs11847697, rs11911017, rs12175867, rs12214416, rs12445022, rs12463617, rs1250229, rs12524865, rs12597579, rs12603327, rs12692735, rs12718465, rs12740374, rs12801636, rs12932445, rs12936587, rs12970066, rs130071, rs13078807, rs1317507, rs13209747, rs1321309, rs13306194, rs13359291, rs1344653, rs1351525, rs13723, rs1378942, rs1412444, rs1421085, rs148910227, rs1496653, rs151193009, rs1514175, rs1535500, rs1552224, rs1555543, rs1563788, rs1591805, rs16849225, rs16858082, rs16986953, rs16990971, rs16999793, rs17030613, rs17035646, rs17080102, rs17087335, rs17135399, rs17249754, rs173396, rs17358402, rs17381664, rs174547, rs17465637, rs17477177, rs17514846, rs17612742, rs17678683, rs17695224, rs1800588, rs181360, rs1861411, rs1868673, rs1870634, rs1887320, rs1892094, rs191835914, rs1976041, rs2000999, rs200990725, rs2021783, rs2057291, rs2066714, rs2068888, rs2075260, rs2075291, rs2107595, rs2128739, rs2144300, rs2145598, rs2156552, rs216172, rs2200733, rs2213732, rs2229383, rs2230808, rs2237896, rs2240736, rs2268617, rs2297991, rs2303790, rs2328223, rs2383208, rs2531995, rs2535633, rs2571445, rs2575876, rs261967, rs2782980, rs2815752, rs2819348, rs2820443, rs2925979, rs2954029, rs29941, rs3120140, rs3129853, rs3130501, rs326214, rs351855, rs35332062, rs35337492, rs35444, rs36096196, rs3775058, rs3785100, rs3809128, rs3827066, rs3846663, rs3887137, rs4129767, rs4148008, rs4266144, rs4302748, rs4377290, rs4409766, rs4410190, rs4420638, rs4468572, rs459193, rs4593108, rs4613862, rs46522, rs4713766, rs4719841, rs4731420, rs4735692, rs4752700, rs4766228, rs4776970, rs4788102, rs4812829, rs4821382, rs4836831, rs4845625, rs4883263, rs4911495, rs4917014, rs4918072, rs499974, rs515135, rs5215, rs556621, rs56062135, rs56289821, rs56336142, rs574367, rs582384, rs590121, rs6038557, rs6065311, rs633185, rs635634, rs6494488, rs651821, rs663129, rs667920, rs6700559, rs671, rs6725887, rs6795735, rs6804922, rs6807945, rs6808574, rs6813195, rs6818397, rs6829822, rs6882076, rs6905288, rs6909752, rs6960043, rs699, rs6997340, rs702485, rs7087591, rs7120712, rs7178572, rs7185272, rs7199941, rs7202877, rs7206541, rs7208487, rs7225581, rs7258445, rs72654473, rs72689147, rs73015714, rs7304841, rs7306523, rs73069940, rs738409, rs740406, rs7499892, rs7500448, rs7503807, rs751984, rs7525649, rs7560163, rs7568458, rs7617773, rs7633770, rs7678555, rs76954792, rs7696431, rs7770628, rs780094, rs7810507, rs7901016, rs7903146, rs7916879, rs7955901, rs7980458, rs7989336, rs80234489, rs8030379, rs8042271, rs806215, rs8090011, rs8108269, rs820429, rs838880, rs867186, rs871606, rs884366, rs885150, rs896854, rs897057, rs9266359, rs9268402, rs9299, rs9319428, rs9349379, rs9357121, rs9367716, rs9376090, rs9390698, rs944172, rs9470794, rs9473924, rs9505118, rs9534262, rs9552911, rs9568867, rs9593, rs9663362, rs9687065, rs975722, rs9810888, rs9815354, rs9818870, rs9828933, rs9892152, and rs9970807.

2. The method according to claim 1, wherein the individual's information further comprises information on one or more of BP-associated single nucleotide polymorphism loci, BMI-associated single nucleotide polymorphism loci, DM-associated single nucleotide polymorphism loci, TC-associated single nucleotide polymorphism loci, and Stroke-associated single nucleotide polymorphism loci:
BP-associated single nucleotide polymorphism loci: rs10051787, rs11651052, rs12037987, rs1275988, rs12999907, rs13041126, rs13143871, rs1558902, rs16896398, rs174546, rs17843768, rs1799945, rs391300, rs4336994, rs4722766, rs507666, rs6825911, rs7213603, rs7405452, rs880315, and rs93138;
BMI-associated single nucleotide polymorphism loci: rs11257655, rs11604680, rs1470579, rs1982963, rs6545814, and rs888789;
DM-associated single nucleotide polymorphism loci: rs10010670, rs10160804, rs1029420, rs1037814, rs1052053, rs10830963, rs10886471, rs10923931, rs11067763, rs11624704, rs11660468, rs117601636, rs1211166, rs12229654, rs12242953, rs12549902, rs12571751, rs1260326, rs12679556, rs12946454, rs13233731, rs13266634, rs13342232, rs1334576, rs1359790, rs1436953, rs1532085, rs1575972, rs16927668, rs16967013, rs17301514, rs17517928, rs17609940, rs17791513, rs17843797, rs1801282, rs1832007, rs2028299, rs2074158, rs2075423, rs2081687, rs2123536, rs2245019, rs2258287, rs2261181, rs2296172, rs2334499, rs243019, rs2487928, rs2642442, rs273909, rs2783963, rs2796441, rs2820315, rs2861568, rs2972146, rs3213545, rs340874, rs35879803, rs368123, rs3774472, rs3791679, rs3810291, rs3861086, rs3918226, rs3936511, rs4142995, rs42039, rs4275659, rs4458523, rs4757391, rs4765773, rs4846049, rs4923678, rs55783344, rs579459, rs58542926, rs6093446, rs634501, rs67156297, rs67839313, rs6825454, rs6831256, rs6871667, rs6878122, rs6909574, rs6984210, rs702634, rs7107784, rs7116641, rs7258189, rs7403531, rs748431, rs7528419, rs7610618, rs7616006, rs769449, rs78169666, rs7897379, rs7917772, rs79223353, rs79548680, rs820430, rs840616, rs9309245, rs9512699, rs9591012, and rs984222;
TC-associated single nucleotide polymorphism loci: rs10401969, rs10889353, rs11136341, rs117711462, rs12027135, rs12453914, rs12927205, rs13115759, rs1367117, rs1495741, rs16844401, rs17122278, rs181359, rs2000813, rs2244608, rs2302593, rs247616, rs4883201, rs5996074, rs7134594, rs7258950, rs737337, rs7965082, and rs964184;
Stroke-associated single nucleotide polymorphism loci: rs10203174, rs1050362, rs10947231, rs11634397, rs11957829, rs12500824, rs12607689, rs13702, rs1424233, rs1467605, rs1508798, rs16933812, rs17080091, rs17608766, rs180327, rs1878406, rs2075650, rs2107732, rs2237892, rs2295786, rs246600, rs2625967, rs2758607, rs2972143, rs34008534, rs35419456, rs376563, rs4471613, rs4724806, rs4777561, rs4939883, rs60154123, rs6544713, rs7136259, rs7193343, rs73596816, rs736699, rs7859727, rs7947761, and rs832552;
preferably, the individual's information further comprises clinical risk factors;
preferably, the individual is from an East Asian population.

3. The method according to claim 1, further comprising:
obtaining a genetic risk score based on the information of the single nucleotide polymorphism loci by the following equation:
$$Genetic risk score = \sum β i \times Ni$$
wherein βi is an effect size of the i^{th} SNP, and Ni is the number of effect alleles of the i^{th} SNP carried by the individual;
preferably, the effect sizes of the SNP are shown in Table 4;
further preferably, the higher the genetic risk score, the higher the individual's risk of developing coronary artery disease is.

4. A device for evaluating a risk of developing coronary artery disease, comprising a detection unit and a data analysis unit, wherein:
the detection unit is used for detecting a sample from an individual to obtain information of an individual to be tested and providing detection results; wherein the information of the individual is the individual's information defined in claim 1; and
the data analysis unit is used for analyzing and processing the detection results from the detection unit to calculate the genetic risk score of the individual to be tested.

5. The device for evaluating the risk of developing coronary artery disease according to claim 4, wherein the analyzing and processing of the detection results from the detection unit by the data analysis unit comprises: assigning weighting factors to the detection results of the single nucleotide polymorphism loci to calculate the genetic risk score of the individual to be tested;
preferably, the data analysis unit comprises:
a preprocessing module for normalizing the detection results of the single nucleotide polymorphism loci;
a calculation module for substituting the normalized detection results of the single nucleotide polymorphism loci into the following evaluation model to obtain a genetic risk score for the individual to be tested:
$$Genetic risk score = \sum β i \times Ni$$
wherein βi is an effect size of the i^{th} SNP, and Ni is the number of effect alleles of the i^{th} SNP carried by the individual;
preferably, the effect sizes of the SNP are shown in Table 4;
further preferably, the higher the genetic risk score, the higher the individual's risk of developing coronary artery disease is.

6. The device for evaluating the risk of developing coronary artery disease according to claim 5, wherein the data analysis unit further comprises a clinical factor processing module for obtaining a 10-year cardiovascular and cerebrovascular risk score by China-PAR of the individual to be tested;
preferably, the calculation module is also used to further combine the genetic risk score with the clinical risk score to evaluate the 10-year incidence risk and/or lifetime risk information for coronary artery disease.

7. The device for evaluating the risk of developing coronary artery disease according to claim 6, wherein the data analysis unit further comprises:
a matrix input module for receiving a plurality of the normalized detection results output from the preprocessing module, and inputting the normalized detection results in a matrix form into the calculation module;
preferably, the data analysis unit further comprises:
an output module for receiving the genetic risk score and/or the 10-year incidence risk and/or the lifetime risk information for coronary artery disease output from the calculation module, and outputting it as a diagnostic classification result.

8. The device for evaluating the risk of developing coronary artery disease according to claim 4, wherein the device is a computer device comprising a memory, a processor, and a computer program stored in the memory and runnable on the processor, wherein when the processor executes the computer program, the device obtains an evaluation result of a risk of developing coronary artery disease of an individual based on information of the individual to be tested;
preferably, wherein the process of obtaining an evaluation result of a risk of developing coronary artery disease of an individual based on information of the individual to be tested comprises: assigning weighting factors to the detection results of the single nucleotide polymorphism loci to calculate a genetic risk score of the individual to be tested; wherein the genetic risk score is a result obtained according to the following evaluation model:
$$Genetic risk score = \sum β i \times Ni$$
wherein βi is an effect size of the i^{th} SNP, and Ni is the number of effect alleles of the i^{th} SNP carried by the individual;
preferably, the effect sizes of the SNP are shown in Table 4;
further preferably, the higher the genetic risk score, the higher the individual's risk of developing coronary artery disease is.

9. A method for establishing a comprehensive polygenic risk score for coronary artery disease, the method comprising the steps of:
(1) screening SNPs to create a collection of single nucleotide polymorphism loci (SNPs) associated with coronary artery disease and/or coronary artery disease-related phenotypes; where the coronary artery disease-related phenotypes include blood pressure, type 2 diabetes, blood lipids, obesity, and stroke;
(2) performing genotyping based on the single nucleotide polymorphism loci in step (1);
(3) extracting the risk alleles, effect sizes, and P values respectively of the measured SNPs corresponding to a plurality of subphenotypes from the results of a genome-wide association study, and establishing a subphenotypic PRS for each subphenotype, the plurality of subphenotypes preferably including coronary artery disease, body mass index, blood pressure, type 2 diabetes, total cholesterol, low density lipoprotein cholesterol, triglycerides, high density lipoprotein cholesterol, and stroke; preferably, wherein a plurality of candidate subphenotypic PRSs are established separately for each subphenotype and screened for the best subphenotypic PRS;
(4) determining the weight of each subphenotypic PRS;
(5) converting the weights of the subphenotypic PRS into weights at the SNP level;
(6) establishing a comprehensive polygenic risk score metaPRS for coronary artery disease.

10. The method according to claim 9, wherein single nucleotide polymorphism loci having a genome-wide significant association with blood pressure include: a single nucleotide polymorphism locus having a genome-wide significant association with systolic blood pressure, a single nucleotide polymorphism locus having a genome-wide significant association with diastolic blood pressure, a single nucleotide polymorphism locus having a genome-wide significant association with pulse pressure, a single nucleotide polymorphism locus having a genome-wide significant association with mean arterial pressure, and a single nucleotide polymorphism locus having a genome-wide significant association with hypertension; single nucleotide polymorphism loci having a genome-wide significant association with obesity include: a single nucleotide polymorphism locus having a genome-wide significant association with body mass index, a single nucleotide polymorphism locus having a genome-wide significant association with waist circumference, and a single nucleotide polymorphism locus having a genome-wide significant association with waist-to-hip ratio; and single nucleotide polymorphism loci having a genome-wide significant association with blood lipid include: a single nucleotide polymorphism locus having a genome-wide significant association with total cholesterol, a single nucleotide polymorphism locus having a genome-wide significant association with low density lipoprotein cholesterol, a single nucleotide polymorphism locus having a genome-wide significant association with triglycerides, and a single nucleotide polymorphism locus having a genome-wide significant association with high density lipoprotein cholesterol.

11. The method according to claim 9, wherein the comprehensive polygenic risk score for coronary artery disease is for evaluating the risk of developing coronary artery disease in an East Asian population;
preferably, a cohort population for the genotyping in step (2) is an East Asian population;
more preferably, the genotyping is performed using a multiplex polymerase chain reaction targeted amplicon sequencing technology.

12. The method according to claim 9, wherein:
in step (3), the process of establishing a PRS for each candidate subphenotype includes:
setting up multiple SNP groups on the basis of the extracted P-values, and for each group of SNPs, pruning according to r²<0.2 based on the cohort population data using the clumping command of the PLINK software to obtain multiple SNP combinations;
using genotype data, weighting and summing up the number of SNP risk alleles (0, 1, or 2) according to their corresponding effect sizes, to establish a plurality of candidate PRSs incorporating different SNP combinations, evaluating the correlation of these candidate PRSs with coronary artery disease using a logistic regression modeling, and selecting the score with the largest odds ratio (OR) as the best subphenotypic PRS;
preferably, the process of determining the weight of each subphenotypic PRS in step (4) comprises:
converting each subphenotypic PRS into normalized scores with a mean of 0 and a standard deviation of 1;
using a training set, putting each of the normalized subphenotypic PRSs and the covariates to be adjusted together into an elastic net logistic regression model, and selecting the model with the highest AUC as the final model from which the coefficients of each PRS (β₁ . . . βₙ) are obtained as weights;
preferably, the process of converting the weights of the subphenotypic PRS into weights at the SNP level in step (5) is performed according to the following model:
$$βsnp\_i = \frac{{β}_{1}}{{σ}_{1}} {α}_{j 1} + … + \frac{{β}_{n}}{{σ}_{n}} {α}_{jn}$$
wherein σ₁, . . . , σᵢ is the standard deviation of each subphenotypic PRS in the training set, αⱼ₁, . . . , αⱼₙ is the effect size of the i^{th} SNP corresponding to each subphenotype, and if a SNP is not included in the k^{th} score, the effect size αⱼₖ of that SNP is set to 0;
preferably, in step (6), the established comprehensive polygenic risk score metaPRS for coronary artery disease is:
$$metaPRS = \sum βsnp\_i \times Ni$$
wherein, βsnp_i is the effect size of the i^{th} SNP, and Ni refers to the number of effect alleles of the i^{th} SNP carried by the individual.

13. The method according to claim 9, wherein by using the 20th and 80th percentiles of the metaPRS of all individuals in the cohort population as cut-offs, the individual is categorized into a population having a low, medium, or high risk of genetic incidence of coronary artery disease.

14. A device for establishing a comprehensive polygenic risk score for coronary artery disease, comprising:
a genotyping module for genotyping each SNP in the collection of single nucleotide polymorphism loci as defined in claim 9;
a subphenotypic PRS establishment module, for extracting the risk alleles, effect sizes, and P values respectively of the measured SNPs corresponding to a plurality of subphenotypes from the results of the genome-wide association study, and establishing a subphenotypic PRS for each subphenotype, wherein the plurality of subphenotypes include coronary artery disease, body mass index, blood pressure, type 2 diabetes, total cholesterol, low density lipoprotein cholesterol, triglycerides, high density lipoprotein cholesterol, and stroke;
a model training module for determining the weight of each subphenotypic PRS in a training set; and
a metaPRS establishment module for converting the weights of the subphenotypic PRS into weights at the SNP level and establishing a comprehensive polygenic risk score metaPRS for coronary artery disease;
preferably, the metaPRS establishment module is further used to evaluate the function of the established metaPRS in the prediction and stratification of the risk of developing coronary artery disease.

15. The device for establishing a comprehensive polygenic risk score for coronary artery disease according to claim 14, wherein the device is a computer device comprising a memory, a processor, and a computer program stored in the memory and runnable on the processor, wherein when the processor executes the computer program, the device evaluates a risk of developing coronary artery disease in an individual by using the comprehensive polygenic risk score metaPRS for coronary artery disease.
